SEQUENCE LISTING (8 sequences)

SEQ ID NO: 1 - DNA, 21 bp, synthetic construct
  source 1..21: mol_type = other DNA
atttggaggacatcccagacc

SEQ ID NO: 2 - DNA, 21 bp, synthetic construct
  source 1..21: mol_type = other DNA
ggtctggccaaatctgttacg

SEQ ID NO: 3 - DNA, 20 bp, synthetic construct
  source 1..20: mol_type = other DNA
ctgtgatgtggacgtgtctt

SEQ ID NO: 4 - DNA, 20 bp, synthetic construct
  source 1..20: mol_type = other DNA
ctccgctggtgtaatccttc

SEQ ID NO: 5 - DNA, 20 bp, synthetic construct
  source 1..20: mol_type = other DNA
atggccgacatatgcaagaa

SEQ ID NO: 6 - DNA, 20 bp, synthetic construct
  source 1..20: mol_type = other DNA
gcatgtgcatcatcatctgg

SEQ ID NO: 7 - DNA, 20 bp, synthetic construct
  source 1..20: mol_type = other DNA
ttcacaacctggagcattca

SEQ ID NO: 8 - DNA, 21 bp, synthetic construct
  source 1..21: mol_type = other DNA
tcacttcttcactggtcatgt

(19) United States
(12) Patent Application Publication
(10) Pub. No.: US 2025/0051844 A1
(43) Pub. Date: Feb. 13, 2025

(54) METHOD FOR MEASURING GENE EXPRESSION OF SINGLE CELL SUBPOPULATION, RELATED KIT, AND APPLICATION

(71) Applicants: Cytomics Limited, Kowloon (CN); The Chinese University of Hong Kong, Shatin, New Territories (CN)

(72) Inventors: Dan Huang, Shatin, New Territories (CN); Kwong Sak Leung, Shatin, New Territories (CN); Leung Sang Nelson Tang, Kowloon (CN)

(21) Appl. No.: 18/719,791

(22) PCT Filed: Nov. 7, 2022
(86) PCT No.: PCT/CN2022/130345

§ 371 Date: Jun. 13, 2024

(30) Foreign Application Priority Data
Dec. 17, 2021 (CN) 202111549870.0

(51) Int. Cl. C12Q 1/6883; C12Q 1/689

(52) U.S. Cl. CPC C12Q 1/6883; C12Q 1/689; C12Q 2600/158

(57) ABSTRACT

The present application provides a peripheral blood sample analysis method and a use of a reagent component for measuring the abundance of a gene transcript in the preparation of a kit for the peripheral blood sample analysis method. The present application also provides a kit comprising the reagent component for quantifying the abundance of the gene transcript and a use of the reagent component for quantifying the abundance of the gene transcript in the preparation of a kit or drug for differentiating and triage of patients having abnormal body temperature.

### TECHNICAL FIELD

The present invention relates to the field of biological detection and analysis, and in particular, to a method for measuring gene expression of a single cell subpopulation, related kit and application.

### BACKGROUND OF INVENTION

The detection and analysis of peripheral blood is an important aspect of medical examination. The peripheral blood is composed of various leukocyte subpopulations, such as neutrophils, lymphocytes, and monocytes (also known as mononuclear leukocytes, mononuclear white blood cells, mononuclear cells, or mononuclear spheres), and thus is a cell-mixture sample. Gene expression of a single cell subpopulation or a single cell-type is a useful biomarker. However, it cannot be detected directly from a cell-mixture sample of peripheral blood by existing methods. In order to obtain gene expression levels of single cell subpopulations, conventional methods require prior isolation of subpopulations of specified cell types. Recently, one another method, which is termed as single cell RNA-sequencing (abbreviated as scRNA-seq), has also made it possible to obtain gene expression information of single cells. The single-cell RNA sequencing generates gene expression data for each cell using expensive equipment and reagents. However, due to the high cost, this technique is generally only used in researches and is not suitable for clinical applications on a large scale.

Several methods can directly determine informative genes of selected single cell subpopulations (single cell-type informative genes) from a cell-mixture sample without isolating target cell subpopulations. They also avoid using expensive equipment of single cell RNA-seq. For details, references can be made to patent document CN103764848B (filed on Jul. 23, 2012, and published on Apr. 30, 2014), and U.S. Pat. No. 9,589,099B2 (filed on Jul. 20, 2012, and published on Mar. 7, 2017). This new detection method is termed as “Direct Leukocyte Subpopulation Transcript Abundance Assay” or “Direct LS-TA Assay” for short. Unlike scRNA-seq, in which sequencing is performed on each cell to detect the transcript abundance therein, the Direct Leukocyte Subpopulation Transcript Abundance Assay is aimed to assess the average gene expression of all cells of the same type of cells (e.g., all B-lymphocytes) from a single cell subpopulation. However, previous methods (such as those disclosed in CN103764848B and U.S. Pat. No. 9,589,099B2) have not focused on the cell subpopulation of monocytes.

Fever is a common clinical symptom. However, there are many causes of fever, and important clinical diagnoses can be broadly divided into several broad categories: (1) bacterial infections, such as pneumonia or other infections caused by bacteria such as staphylococcus, streptococcus, meliodiosis, or haemophilus: (2) viral infections caused by influenza virus, RSV, etc.: (3) pulmonary tuberculosis, such as tuberculosis and active pulmonary tuberculosis, but excluding latent tuberculosis (Latent TB) infection; and (4) autoimmune diseases, such as systemic lupus erythematosus (SLE).

Various leukocyte subpopulations in peripheral blood respond differently to different diseases. Although they all cause fever symptoms, their cellular responses differently to various pathogens or etiologies. While there are many clinical markers and symptoms (which are commonly used by doctors) that can help to differentiate the causes of fever, it is helpful for the work of doctors if in addition to clinical symptoms, there are assays for symptom identification or differentiation so as to differentiate patients according to the major types of etiologies. Existing assays, including those on various serum proteins (e.g., CRP complements), inflammatory response proteins such as cytokines, complete blood count (blood routine test for complete blood count), erythrocyte sedimentation rate (ESR) and the like, are not specific markers, and the final clinical judgment and differentiation still require the experience of doctors to make the decision. At the same time, these existing clinical tests basically do not pay attention to genetic changes or functional indications of various component leukocyte subpopulations in the peripheral blood. For example, various serum tests actually only use the serum that is kept after all the leukocytes are directly separated and discarded. The blood routine test only focuses on the cell counts or proportional cell counts of various leukocyte subpopulations in peripheral blood, and cannot reflect any change in function of the various leukocyte subpopulations.

However, among the above four major types of causes of fever, bacterial infections and pulmonary tuberculosis are of particular clinical significance, and they require immediate targeted treatment or isolation of patients. As for bacterial infections, administration of prescribed antibiotics needs to be performed as early as possible so as to suppress bacterial proliferation and control the diseases. As for pulmonary tuberculosis, it is necessary to isolate the patients (to avoid infecting others) and administrate prescribed anti-tuberculosis drugs as early as possible. For the remaining two major groups, the urgency of differentiation is less pressing than for the two groups of bacterial infections and tuberculosis. Therefore, if there are biomarkers that can differentiate these two major groups, it will play a very important role in clinical differentiation of emergency cases and triage of patients. After the completion of the human genome map in 2000, most of the genes have been identified. In addition, there are various methods to detect many genes in a large scale (e.g., microarray, qPCR, RNA-seq, and digital PCR). Researchers have also attempted to detect genetic alterations in whole blood (WB) or other peripheral blood samples (such as peripheral blood mononuclear cell samples, PBMCs) for various diseases (Berry et al. 2010; Blankley, Graham, Levin, et al. 2016; Gupta et al. 2020).

However, methods that have been used so-far focus on identifying differential expression genes (DEGs), using various statistics, machine learning and algorithms and mathematical methods to analyze the differences in expression profiles in genome expression data. Therefore, they result in a very long list of genes, which comprises the degrees of expression differences of the list of individual genes between the disease group and the control group, with genes having larger differences then being used as biomarkers (Sweeney, Wong, and Khatri 2016; Tsalik et al. 2016; McClain et al. 2021; Lydon et al. 2019; Tsao et al. 2020). These studies have also been the subject of recent review articles (Tsao et al. 2020; Holcomb et al. 2017; Gliddon et al. 2018).

Such research methods often result in a very long list of genes, including many differentially expressed genes, ranging from dozens to hundreds (Tsalik et al. 2016; Zaas et al. 2013; Lydon et al. 2019; Mejias et al. 2013; Sweeney, Wong, and Khatri 2016; McClain et al. 2021; Mahajan et al. 2016). As the number of genes that need to be detected increases, the feasibility of clinical use becomes limited. In addition, these tests also require the use of special or expensive equipment. These limitations constraint the utility of genes typically identified from differential expression analysis. A recent trend is to screen out several genes with high discriminative performance from a lengthy list of differential genes for clinical testing. Therefore, schemes that use the detection of expression of genes (the number of which is within three, four, or ten) to differentiate the causes of infection have recently emerged (Sweeney, Wong, and Khatri 2016; Sampson et al. 2017; Herberg et al. 2016; Gómez-Carballa et al. 2021; 2019; Gliddon et al. 2021). Many of these schemes using a small number of genes are based on the use of interferon stimulated genes (ISG), since viral infection stimulates interferon secretion and turns on interferon stimulated genes (e.g., ISG15, OASL, IFI27, IFI44L, IFIT1 and IFITM3) where the responses of these ISG genes are thus specific to viral infection. In contrast, not much is known about genes whose expression changes specifically in bacterial infections.

These above-mentioned studies for differentially expressed genes have not paid attention to potential changes in cell counts in cell-mixture samples such as whole blood. Another significant shortcoming and limitation of these methods is that they do not account for changes in cell counts of various leukocyte subpopulations in peripheral blood. As many genes are expressed by more than one leukocyte subpopulations, changes in the cell counts of the various leukocyte subpopulations in peripheral blood can lead to changes in total expression amounts of many genes in whole blood. Due to this confounding factor, many peripheral blood biomarkers discovered at early stage cannot be confirmed by subsequent studies, and they may be false positives or caused by changes in the cell counts of various leukocyte subpopulations in peripheral blood, and cannot reflect specific responses in specific leukocyte subpopulations to diseases.

In addition, some studies have tried to categorize the differentially expressed genes into various module groups (Blankley, Graham, Levin, et al. 2016; Rinchai et al. 2021: Chaussabel 2015). However, in the end, it is necessary to analyze multiple modules (such as dozens of modules), and each module also comprises many genes. Special software is needed to perform such analysis (Rinchai et al. 2021), which cannot achieve the effect of simple and easy differentiation of patients.

Other studies began to focus on the subject of cell counts of various cell subpopulations in cell-mixture samples and its solution, and mathematical deconvolution methods have been developed. Using data from whole gene expression profiles, cell counts or proportional cell counts of various cell subpopulations in a cell-mixture sample are first inferred. Then, whether there is any difference in the average expression of each gene in the disease group and the control group is calculated (Shen-Orr et al. 2010; Newman et al. 2015; Nadel et al. 2021). This series of computing solutions are widely used in gene expression profiling of cancer tissues, and are used to calculate the proportional cell counts of cancer cells and various leukocyte subpopulations in cancer tissue samples, so as to predict the prognosis for cancer. However, these methods always focus on the various proportional cell counts, and cannot directly derive the expression levels of individual genes for various leukocyte subpopulations in the cell-mixture sample. Therefore, it is still not possible to determine the expression level of each gene in an individual sample. Another disadvantage of these methods is that they need to be provided with data on the whole gene expression profiles, and therefore can only be applied to the results obtained from platforms that detect gene expression profiles (e.g., microarray and RNA-seq) (Nadel et al. 2021). These platforms are also now widely used in research work, but it is clearly not feasible to use them for routine clinical differentiation. For example, the simplest genetic detection process by microarray takes two to three working days, and the RNA-seq takes an even longer (up to 1 week) time for detection. In short, although these platforms show good research utilities, they cannot be used in clinical applications on a large scale at this stage due to cost and time-consuming issues.

Therefore, a new simple and rapid method for analyzing peripheral blood is needed, so that a febrile patient can be quickly differentiated and triaged.

### SUMMARY OF INVENTION

In general, provided in the present application is an analytical method, a corresponding kit and use. The method can be used to directly evaluate expression levels of monocyte-specific genes by directly measuring the transcript abundance (TA) of the monocyte cell-type informative genes in various cell-mixture samples (such as peripheral blood, including WB and PBMC), thereby avoiding the prior isolation of monocytes and the need for expensive equipment for single-cell RNA-sequencing. Biomarkers obtained by this method can be used in various clinical applications, such as differentiation of causes of fever.

Specifically, in a first aspect, the present application provides a method for analyzing a peripheral blood sample, comprising measuring, in the peripheral blood sample, the transcript abundance of a single cell subpopulation target gene and the transcript abundance of a single cell subpopulation reference gene in a peripheral blood sample, wherein the single cell subpopulation target gene is selected from at least one of those shown in Table 2-2, and the single cell subpopulation reference gene is selected from PSAP or CTSS.

In some preferred embodiments, the single cell subpopulation is monocytes.

In particular embodiments, the single cell subpopulation target gene is selected from one or more of VNN1, CYP1B1, NLRC4, PFKFB3, LILRA5, NFKBIZ, CALHM6, WARS1, ATF3, IFITM3, IFI44L and IFI30.

In a second aspect, the present application provides use of a reagent component for measuring the transcript abundances of genes in the preparation of a kit for use in a method for analyzing a peripheral blood sample, wherein the method includes the quantification of a single cell subpopulation target gene and the transcript abundance of the single cell subpopulation reference gene in peripheral blood samples, wherein the single cell subpopulation target gene is selected from at least one of those shown in Table 2-2, and the single cell subpopulation reference gene is selected from PSAP or CTSS.

In some preferred embodiments, the single cell subpopulation is monocytes.

In particular embodiments, the single cell subpopulation target gene is selected from one or more of VNN1, CYP1B1, NLRC4, PFKFB3, LILRA5, NFKBIZ, CALHM6, WARS1, ATF3, IFITM3, IFI44L and IFI30.

The method for analyzing a peripheral blood sample described above comprises the following steps of:
a). obtaining a peripheral blood sample;
b). measuring the transcript abundance of the single cell subpopulation target gene in a peripheral blood sample to obtain a first amount;
c). measuring the transcript abundance of the single cell subpopulation reference gene in a peripheral blood sample to obtain a second amount; and
d). calculating a biomarker parameter, wherein said parameter is a relative value of said first amount to said second amount.

In some embodiments, the method further comprises comparing the relative value to a cutoff value.

In a third aspect, the present application provides a kit comprising a reagent component for quantifying the transcript abundance of genes, wherein the genes are selected from at least one of target genes shown in Table 2-2 and at least one of reference genes shown in Table 2-1.

In a fourth aspect, the present application provides use of a reagent component for quantifying the transcript abundance of genes in the preparation of a kit for differentiating and triaging a patient having abnormal body temperature or medicament, wherein the genes are selected from at least one of target genes shown in Table 2-2 and at least one of reference genes shown in Table 2-1.

In the kit of the third aspect or the use of the fourth aspect, the target genes are a combination of at least one gene selected from group (1) of genes and at least one gene selected from group (2) of genes: a combination of at least one gene selected from group (1) of genes and at least one gene selected from group (3) of genes; a combination of at least one gene selected from group (2) of genes and at least one gene selected from group (3) of genes: or a combination of at least one gene selected from group (1) of genes, at least one gene selected from group (2) of genes and at least one gene selected from group (3) of genes:
(1) VNN1, CYP1B1, NLRC4, PFKFB3, LILRA5, NFKBIA, NFKBIZ and NAIP;
(2) CALHM6, WARS1, GADD45B, NR4A1, SGK1, ATF3 and TCN2; and
(3) IFITM3, IFI44L and IFI30.

In some embodiments, the patient having abnormal body temperature is a febrile patient. In some preferred embodiments, the patient having abnormal body temperature is a patient with a bacterial infection, a patient with a viral infection, a patient with a pulmonary tuberculosis or a patient with an autoimmune disease. In some more preferred embodiments, the patient with a viral infection is a patient with an influenza virus infection, the patient with a pulmonary tuberculosis is a patient with active pulmonary tuberculosis, and the patient with an autoimmune disease is a patient with systemic lupus erythematosus.

In some embodiments, one or more genes in group (1) of genes are used to differentiate a patient with a bacterial infection, one or more genes in group (2) of genes are used to differentiate a patient with a pulmonary tuberculosis, in particular a patient with active pulmonary tuberculosis, and/or one or more genes in group (3) of genes are used to differentiate a patient with a viral infection or a patient with an autoimmune disease.

In some embodiments, the combination of at least one gene selected from group (1) of genes and at least one gene selected from group (2) of genes may be used to differentiate whether the patient having abnormal body temperature is a patient with a bacterial infection or a patient with active pulmonary tuberculosis. In some preferred embodiments, a combination of VNN1 selected from group (1) of genes and CALHM6 selected from group (2) of genes may effectively differentiate a patient with a bacterial infection and a patient with a pulmonary tuberculosis.

In some embodiments, the combination of at least one gene selected from group (1) of genes and at least one gene selected from group (3) of genes may be used to differentiate whether the patient having abnormal body temperature is a patient with a bacterial infection, a patient with a viral infection or a patient with an autoimmune disease.

In some embodiments, the combination of at least one gene selected from group (2) of genes and at least one gene selected from group (3) of genes may be used to differentiate whether the patient having abnormal body temperature is a patient with active pulmonary tuberculosis, a patient with a viral infection, or a patient with an autoimmune disease.

In some embodiments, the combination of at least one gene selected from group (1) of genes, at least one gene selected from group (2) of genes and at least one gene selected from group (3) of genes may be used to differentiate whether the patient having abnormal body temperature is a patient with a bacterial infection, a patient with active pulmonary tuberculosis, a patient with a viral infection or a patient with an autoimmune disease.

In some preferred embodiments, the patient having abnormal body temperature is a pediatric patient with Kawasaki disease or a pediatric patient with a virus infection. In some embodiments, the combination of at least one gene selected from group (1) of genes and at least one gene selected from group (3) of genes may be used to differentiate whether the patient having abnormal body temperature is a patient with Kawasaki disease or a patient with a viral infection.

In some embodiments, one or more genes in group (1) of genes are used to differentiate a pediatric patient with Kawasaki disease.

In particular embodiments, one or more genes in group (1) of genes may be used to differentiate a pediatric patient with Kawasaki disease and a pediatric patient with a similar symptom but having a virus infection.

In preferred embodiments, the combination of VNN1 selected from group (1) of genes, WARS1 selected from group (2) of genes, and IFI44L selected from group (3) of genes can effectively differentiate four categories of febrile diseases (i.e. bacterial infections, pulmonary tuberculosis, viral infections and autoimmune diseases).

In the kit of the third aspect or the use of the fourth aspect, the reagent component comprises primers, and the sequences of the primers are set forth in any one of SEQ ID NOs: 1-8.

In some preferred embodiments, the gene is originated from a peripheral blood sample. In some more preferred embodiments, the gene is originated from monocytes in the peripheral blood sample.

### BRIEF DESCRIPTION OF DRAWINGS

The present invention will be further described in conjunction with the drawings, in which:

FIG. 1 shows the overall workflow of a method for directly measuring the gene expression of monocytes from a peripheral blood sample. Expression data from already isolated monocyte samples and cell-mixture samples (e.g., PBMC) are first used to determine which genes are characteristic monocyte informative genes (110, 120 in FIG. 1). Then, a reference gene and a target gene are selected from a list of informative genes (130 in FIG. 1). These prerequisite steps are generally carried out by manufacturers or have been disclosed in this application document. When used to triage patients, a new biomarker parameter is calculated from the ratio of the transcript abundance (TA) of the target gene to the transcript abundance of another characteristic informative reference gene of monocytes (such as PSAP or CTSS) to reflect gene expression level of the isolated and purified monocytes (140 in FIG. 1). This new biomarker parameter is termed as “Direct Monocyte Transcript Abundance” (abbreviated as “Direct Monocyte LS-TA”) and can be used to differentiate the etiology of a febrile patient (150 in FIG. 1).

FIG. 2 shows the limitations and disadvantages of an analysis commonly used for differentially expressed genes (DEGs) in a peripheral blood sample. Overall gene expression level is affected by changes in cell counts across various cell subpopulations, which becomes an important confounding factor. For example, the total level of Gene A expression in the peripheral blood sample before infection is 64 transcripts (201 in FIG. 2), assuming that there are three different subpopulations of cells in this sample, i.e., cells represented by squares, diamonds and circles. The number in the cell shape symbol represents the average gene expression level of this type of cells. For example, here we are focusing on square cells, with an average expression level of 8. The total expression level of the three types of cells is 64, which is also the total level of gene A expression measured in this cell-mixture sample. It is assumed that the total level of gene A expression in the blood sample is increased after infection to 96 transcripts (as in the case of A (202 in FIG. 2) or B (203 in FIG. 2)), or the total expression level does not change, i.e., still 64 transcripts (as in the case of C (204 in FIG. 2)). In the cases of A (202 in FIG. 2) and B (203 in FIG. 2), the total expression level of gene A increases, while the expression level of square cells may not change (as in the case of A (202 in FIG. 2), it remains at 8), or increases (as in the case of B (203 in FIG. 2), it increases to 16), indicating that a change in overall gene expression cannot be used to confirm the change in gene expression of a particular type of cells. In the case of C (204 in FIG. 2), the average gene expression of the specific square cells increases to 16, while the total expression level of gene A is unchanged due to the decrease in cell count of the square cells. From the above, the square cells of interest may have different degrees of changes in gene expression, and therefore, the overall expression level of gene transcripts cannot distinguish the changes in gene expression of various types of cells.

FIG. 3 shows the comparison between a traditional method (FIG. 3A) and the method of the present application (FIG. 3B) for measuring the expression abundance of target genes in a cell subpopulation. The traditional method of obtaining gene expression data of a specified cell subpopulation from a cell-mixture sample (e.g., whole blood 301) requires cumbersome experimental steps (302) to isolate that the cell subpopulation (e.g., monocytes, represented by square symbols, 303), and then the gene expression abundance of the cell subpopulation can be measured (310). Although the experimental process is cumbersome, the result of target gene expression abundance in the specified cell subpopulation obtained by the traditional method is generally regarded as the “gold standard” (305). For the “direct monocyte subpopulation transcript abundance” method in the present application (FIG. 3B), gene expressions (320) of the specified cell subpopulation can be determined directly from a cell-mixture sample (e.g. whole blood 301) without the need of cell isolation, which are used to calculate the parameter, i.e., Direct Monocyte Subpopulation Transcript abundance (referred to as “Direct Monocyte LS-TA”, 306). The biomarker parameter (306) has high correlation and transferability with the gold standard (305), and can be a useful biomarker for clinical differentiation or other applications.

FIG. 4 shows a schematic diagram of screening for cell subpopulation-informative genes in the case of the specific cell subpopulation with a specific proportional cell count (for example, monocytes, the proportional cell count of which is set to 20%). If a gene, such as gene A, has an average cellular expression level (422) in an isolated cell sample (412) that is 2.5 times higher than its average cellular expression level (421) in a cell-mixture sample (410) (for the square cell subpopulation, 8/3.2=2.5), then this cell subpopulation (i.e., the square cell subpopulation, 418) contributes 50% of gene A transcripts in the cell-mixture sample (410). This fold-difference is determined by cell proportions and is denoted by “X50” in the present application. The gene expression level (421, 422, 423, 424) is represented by a fraction, wherein the numerator is the total number of transcripts, and the denominator is the number of cells. For example, there are 64 gene A transcripts in the cell-mixture sample (410), which are produced by a total of 20 cells. In this example, the square cell subpopulation is the cell subpopulation of interest, and its X50=2.5 times. Genes with expression level (422) 2.5 times higher in the isolated square cell subpopulation than in the cell-mixture sample (421) are eligible to qualify as informative genes for the square cell subpopulation. In this example, in order to demonstrate the principle, it is assumed that the gene expression levels (423, 424) of other cells (417, 419) are known. In fact, we only need to know the expression levels (421, 422) of the specified cell sample after isolation (412) and the corresponding cell mixture sample (410) to identify the single cell subpopulation informative gene.

FIG. 5 shows a feasible division of labor schema of an embodiment of the present application. Steps 110 to 130 are performed by manufacturers to identify two types of monocyte informative genes, i.e., the target genes (520) and the reference genes (540), and then a kit is prepared for use in clinical differentiation. When used clinically, steps 140 and 150 are performed to measure the transcript expression abundance of at least one target gene (520) and reference gene (540) in peripheral blood, and thus the new biomarker “direct monocyte subpopulation transcript abundance” is calculated and applied clinically to differentiate diseases (150).

FIG. 6 shows the correlation between the results of biomarkers obtained by the “Direct Monocyte LS-TA” assay and the expression of target genes detected in isolated monocytes by the traditional method in the GSE60424 dataset. LYZ, which is a highly expressed gene in monocytes, is selected as the target gene, and CD14 and CTSS identified in the present application (corresponding to FIG. 6A and FIG. 6B, respectively) are respectively used as reference genes in the cell-mixture sample. The X-axis in FIG. 6 shows the determination of gene expression of LYZ in isolated and purified monocytes, using B2M as a conventional housekeeping gene, where the X-axis here is the gold standard. The Y-axis in FIGS. 6A and 6B shows the biomarker parameters of the Direct Monocyte LS-TA in WB, wherein CD14 and CTSS are used as reference genes, respectively. The X-axis in FIGS. 6A and 6B shows the Log(LYZ/B2M) in monocytes after isolation and purification, which is used as the gold standard.

FIG. 7 shows the correlation between the results of biomarkers obtained by the “Direct Monocyte LS-TA” assay and the expression of target genes detected in isolated monocytes by the traditional method in the GSE163605 dataset. VNN1, which is a highly expressed gene in monocytes, is selected as the target gene, and CD14 and PSAP identified in the present application (corresponding to FIG. 7A and FIG. 7B, respectively) are respectively used as reference genes in the cell-mixture sample. The X-axis in FIG. 7 shows the determination of gene expression of VNN1 in isolated and purified monocytes, using B2M as a conventional housekeeping gene, where the X-axis here is the gold standard. The Y-axis in FIGS. 7A and 7B shows the biomarker parameters of the Direct Monocyte LS-TA in PBMC, wherein CD14 and PSAP are used as reference genes, respectively. The X-axis in FIGS. 7A and 7B shows the Log(VNN1/B2M) in monocytes after isolation and purification, which is used as the gold standard.

FIG. 8 shows the correlation between the biomarker parameter of “Direct Monocyte LS-TA” for monocyte informative target genes measured in the cell-mixture sample of peripheral blood and expression levels of the same target genes in monocytes obtained by the traditional method, using PSAP as a monocyte informative reference gene. FIG. 8A shows the VNN1 gene expression in monocytes determined by the method of the present application and the traditional method. The Y-axis is the ratio of VNN1:PSAP determined directly from the cell-mixture sample of peripheral blood (i.e., a Direct Monocyte LS-TA biomarker of VNN1 gene). The X-axis is the gold standard, using the traditional method to detect VNN1 expression after isolation and purification of monocytes, and using a conventional housekeeping gene (B2M) for normalization. As shown in FIG. 8A, there is a good correlation between the two. Evaluation of the performance of other monocyte informative genes using “direct monocyte LS-TA” in peripheral blood is shown in FIG. 8B-8Q, where the genes are CALHM6, ATF3, SIGLEC1, NFKBIZ, NFKBIA, PFKFB3, IFI44L, MERTK, NAIP, CYP1B1, WARS1, GADD45B, SGK1, NR4A1, IFITM3, and NLRC4, respectively. Dataset accession numbers for data sources are shown above FIGS. 8A-8Q. Logarithms used in FIGS. 8A-8Q are natural logarithms.

FIG. 9 shows the correlation between the biomarker parameter of “Direct Monocyte LS-TA” for monocyte informative target genes measured in the cell-mixture sample of peripheral blood and expression levels of the same target genes in monocytes obtained by the traditional method, using the gene CTSS instead of PSAP as a monocyte informative reference gene. FIG. 9A shows the VNN1 gene expression in monocytes determined by the method of the present application and the traditional method. The Y-axis is the ratio of VNN1:CTSS determined directly from the cell-mixture sample of peripheral blood (i.e., the Direct Monocyte LS-TA biomarker of VNN1 gene). The X-axis is the gold standard, using the traditional method to detect VNN1 expression after isolation and purification of monocytes, and using a conventional housekeeping gene (B2M) for normalization. As shown in FIG. 9A, there is a good correlation between the two. The results show that the biomarker parameter of “Direct Monocyte LS-TA” (the ratio of VNN1:CTSS) can fully reflect or replace the VNN1 gene expression level of monocytes obtained by the traditional method using cell isolation steps. Evaluation of the performance of other monocyte informative genes assessed in peripheral blood using “direct monocyte LS-TA” are shown in FIG. 9B-9Q, where the genes are CALHM6, ATF3, SIGLEC1, NFKBIZ, NFKBIA, PFKFB3, IFI44L, MERTK, NAIP, CYP1B1, WARS1, GADD45B, SGK1, NR4A1, NLRC4 and IFITM3, respectively. Dataset accession numbers for data sources are shown above FIGS. 9A-9Q. Logarithms used in FIGS. 9A-9Q are natural logarithms.

FIG. 10A shows the biomarker (i.e., log(VNN1/PSAP)) of expression abundance of log(the “Direct Monocyte LS-TA” of the VNN1 gene) of peripheral blood samples in the GSE154918 (Herwanto et al. 2021) dataset in the control group and the uncomplicated bacterial infection group, respectively. FIG. 10B shows the results after converting the log(VNN1/PSAP) in FIG. 10A to a multiple of median (MoM).

FIG. 11 shows the analysis of the MoM of the “Direct Monocyte LS-TA” for the target gene VNN1 in the control group and the bacterial infection group. In the five datasets analyzed (GSE154918, GSE40012, GSE42026, GSE60244, and GSE63990, respectively), the numbers above the X-axis represent the number of people in the control group and the bacterial infection group, respectively. In each dataset, the MoM results on the Y-axis are natural log-transformed, and therefore, each unit on the Y-axis represents approximately a 2.7-fold difference. For example, in all datasets, the expression of the VNN1 gene in monocytes of patients with bacterial infection is more than 2.7 times higher than the median of that in the control group (the difference in MoM between the two groups in the dataset GSE60244 is the smallest, and the difference on Y axis between the two groups is actually 1).

FIG. 12 shows the receiver operating characteristic (ROC) curve analysis of the discriminative performance of the “Direct Monocyte LS-TA” for use in the determination of VNN1 gene expression in the bacterial infection group.

FIGS. 13A-F show the six additional target genes (i.e., NLRC4, CYP1B1, PFKFB3, LILRA5, NFKBIA, and NFKBIZ, respectively) of monocytes, the expression of which are affected by bacterial infection. Here, the Direct Monocyte LS-TA is calculated using PSAP as a reference gene. For each gene, the difference between MoM of gene expression of monocytes (the Direct Monocyte LS-TA) in peripheral blood between the control group and the bacterial infection group is shown by boxplots (left). The right panel shows the ROC for each gene in differentiating uncomplicated bacterial infections.

FIGS. 14A-F show the differences of the Direct Monocyte LS-TA of the six additional target genes (i.e., NLRC4, CYP1B1, PFKFB3, LILRA5, NFKBIA, and NFKBIZ, respectively) of monocytes between the bacterial infection group and the control group, the expression of which is affected by bacterial infection, and the corresponding ROC analysis results. Here, the Direct Monocyte LS-TA is calculated using CTSS as a reference gene. For each gene, the difference between gene expression of monocytes (the Direct Monocyte LS-TA) in peripheral blood between the bacterial infection group and the control group is shown by boxplots (left). The right panel shows the ROC for each gene in differentiating uncomplicated bacterial infections.

FIG. 15 shows the analysis of MoM of the “Direct Monocyte LS-TA” of the target gene WARS1 in the active pulmonary tuberculosis (TB) group and the control group in the five datasets analyzed (GSE107991, GSE107994, GSE114192, GSE42834, and GSE83456, respectively). In each dataset, the numbers above the X-axis represent the number of people in the control group and the active pulmonary tuberculosis group, respectively. The MoM results on the Y-axis are natural log-transformed, and therefore, each unit on the Y-axis represents approximately a 2.7-fold difference. For example, in the dataset GSE107994, the median difference on the Y axis is about 1, which means that the expression of WARS1 by the “Direct Monocyte LS-TA” in patients is about 2.7 times higher than that of normal people.

FIG. 16 shows the receiver operating characteristic (ROC) curve analysis to determine the discriminative performance of the “Direct Monocyte LS-TA” for use in the determination of the WARS1 gene expression in patients with active pulmonary tuberculosis.

FIGS. 17A-F show the difference of the Direct Monocyte LS-TA of six additional target genes (i.e., CALHM6, GADD45B, SGK1, ATF3, TCN2 and NR4A1, respectively) of monocytes between the active pulmonary tuberculosis group and the control group, the expression of which is affected by active pulmonary tuberculosis, and the corresponding ROC analysis result. Here, the Direct Monocyte LS-TA is calculated using PSAP as a reference gene. For each gene, the difference of the MoM between gene expression of monocytes (the Direct Monocyte LS-TA) in peripheral blood between the control group and the active pulmonary tuberculosis group is shown by boxplots (left). The right panel shows the ROC for each gene in differentiating uncomplicated active pulmonary tuberculosis.

FIG. 18 shows the analysis of the MoM of the “Direct Monocyte LS-TA” of the target gene VNN1 in the Kawasaki disease group and the control group in the three datasets analyzed (GSE73463, GSE73461, and GSE68004, respectively). In each dataset, the numbers above the X-axis represent the number of people in the control group and the Kawasaki disease group, respectively. The MoM results on the Y-axis are natural log-transformed, and therefore, each unit on the Y-axis represents approximately a 2.7-fold difference. For example, in all three datasets, there are statistically significant differences in the VNN1 gene expression of monocytes between the patients in Kawasaki disease group and the median of that in the control group.

FIG. 19 shows the receiver operating characteristic (ROC) curve analysis to determine the discriminative performance of the “Direct Monocyte LS-TA” for use in the determination of the VNN1 gene expression in pediatric patients having Kawasaki disease.

FIG. 20 shows the distribution of the MoM values of the Direct Monocyte LS-TA of VNN1 (shown on the X-axis) and the MoM values of the Direct Monocyte LS-TA of CALHM6 (shown on the Y-axis) in two-dimensional space. FIG. 20A: the patients with bacterial infection are represented by solid circles, and the control group is represented by hollow circles; FIG. 20B: the patients with influenza virus infection are represented by solid circles, and the control group is represented by hollow circles; FIG. 20C: the patients with SLE are represented by solid circles, and the control group is represented by hollow circles; and FIG. 20D: the patients with active pulmonary tuberculosis are represented by solid circles, and the control group is represented by hollow circles.

FIG. 21 shows ROC analysis and confusion matrix and balanced accuracy results for the MoM of the “Direct Monocyte LS-TA” of two genes VNN1 and CALHM6. FIG. 21A shows the ROC analysis for classifying four groups of patients (i.e. patients with bacterial infection, patients with pulmonary tuberculosis, patients with SLE and patients with influenza virus infection) using regions defined by the MoM of the “Direct Monocyte LS-TA” of the two genes VNN1 and CALHM6. FIG. 21B shows the confusion matrix of the four groups of patients in FIG. 21A, wherein the vertical axis shows the actual diagnosis of each group of patients, and the horizontal axis shows the predicted patient grouping based on the two gene differentiation scheme. For example, for 15 patients who indeed have bacterial infections, 12 of them are correctly identified as having bacterial infections by this direct LS-TA protocol, leaving only three patients with bacterial infections being misidentified. Balanced accuracy results are shown below in FIG. 21B.

FIG. 22 shows the differentiation performance of three-dimensional spatial distribution of the Direct Monocyte LS-TA. Using three monocyte gene expression biomarkers (MoMs of “Direct Monocyte LS-TA”), and projecting them onto 3D space, the distribution of the control group and patient having four diseases is shown. The X-axis is the MoM of the Direct Monocyte LS-TA of IFI44L. The Y-axis is the MoM of the Direct Monocyte LS-TA of WARS1. The Z-axis is the MoM of the Direct Monocyte LS-TA of VNN1. Vector labels designate the three axes.

FIG. 23 shows ROC analysis and confusion matrix and balanced accuracy results for the MoM of the “Direct Monocyte LS-TA” of three target genes (i.e., VNN1, WARS1 and IFI44L). FIG. 23A shows the ROC analysis for classifying four groups of patients (i.e. patients with bacterial infection, patients with pulmonary tuberculosis, patients with SLE and patients with influenza virus infection) using regions defined by the MoM of the “Direct Monocyte LS-TA” of the three target genes. FIG. 23B shows the confusion matrix of the four groups of patients, wherein the vertical axis shows the actual diagnosis of each group of patients, and the horizontal axis shows the predicted patient grouping based on the three-gene differentiation scheme. For example, for the 15 patients who indeed have bacterial infections, 12 of them are correctly identified as having bacterial infections by this direct LS-TA protocol, leaving only three patients with bacterial infections being misidentified. Balanced accuracy results are shown below in FIG. 23B.

### DETAILED DESCRIPTION

In a traditional method of obtaining gene expression for particular cells from a peripheral blood sample, that particular leukocyte subpopulation needs to be isolated first and then, gene expression (transcript abundance, TA) detection is performed on that isolated and purified particular cells. As the isolation procedure for the particular cells requires a long time of manual manipulation, it is not suitable for large-scale clinical use. The use of isolating and purifying monocytes first and then quantification of their gene expression to differentiate cancer and other diseases has been described in the prior art (Mazzone, 2018; Buschmann et al. 2017).

There have been studies using various isolation and purification experimental methods for isolating leukocyte subpopulations from peripheral blood of research subjects, and then quantify the gene expression of these cells. These data can be associated with different diseases or used in clinical tests. Transcript abundance of genes in isolated and purified monocytes is used as the gold standard in the present application.

The present application provides a scheme capable of directly measuring the transcript abundance of genes for a specified single cell subpopulation (e.g., monocytes) in cell-mixture samples without isolating the target cell subpopulation. The method is called “Direct Leukocyte Subpopulation Transcript abundance assay”, which is abbreviated as “Direct LS-TA assay”. This method provides a measurement technique for directly determining transcript abundance of selected/specified cell subpopulation from peripheral blood samples without cell isolation. This technique has broad applications and significant advantages in terms of cost. In addition, this method avoids the use of expensive equipment and assays such as those required for single-cell RNA sequencing.

For the direct LS-TA assay, please refer to patents CN103764848B and U.S. Pat. No. 9,589,099B2 for details. These patents proposed this new assay method and its framework scheme, and implemented it in several target genes of lymphocytes and granulocytes to illustrate the feasibility of this scheme. However, the above patent literatures do not focus on monocytes. Specifically, the present application provides direct detection of gene expression in monocytes from peripheral blood without isolating said monocytes.

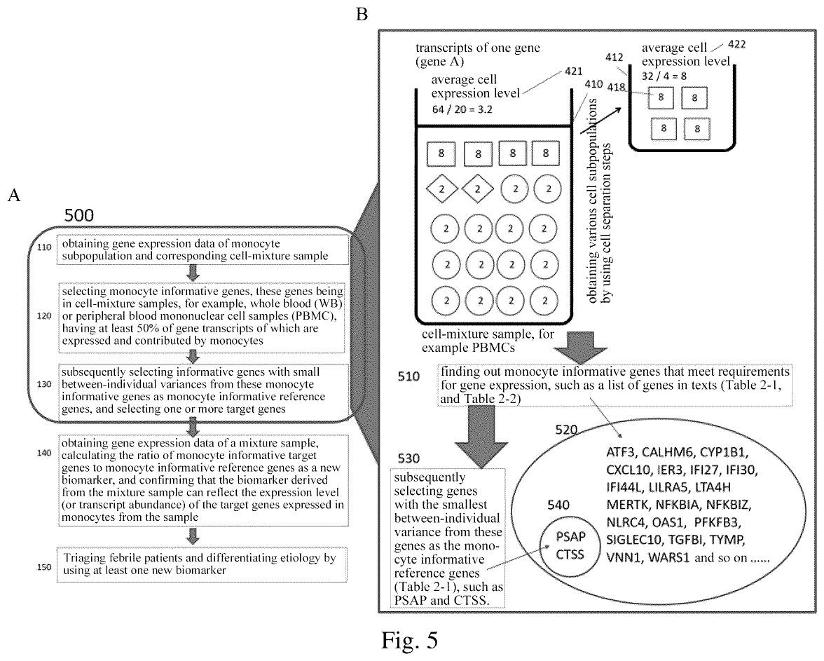

The present application is based on the use of the above scheme in monocytes, and a list of monocyte informative genes that can be used by the Direct Leukocyte Subpopulation Transcript Abundance assay has been derived, and useful target genes and reference genes have been subsequently determined (510, 520, and 530 in FIG. 5), and then transcript abundance of the monocyte subpopulation has been directly determined in whole blood. The ratios of gene expression abundances of these target genes to those of reference genes are good biomarkers, and can be used to differentiate major categories of febrile illness. For example, by performing this test on the peripheral blood samples from febrile patients in the emergency room, the patients can be generally classified, which is helpful for the initial treatment beyond emergency differentiation.

Previous gene expression biomarkers developed by using peripheral blood samples are all based on statistical analysis methods of differential expression genes (DEGs). The expression of each gene is statistically analyzed one by one, and then the gene with the greatest expression difference between different groups is identified as the biomarker. This method ignores the confounding factor of the cell counts of various cell subpopulations and their variations in different diseases. Therefore, variations in these factors will weaken the effectiveness of DEG biomarkers in differentiating diseases.

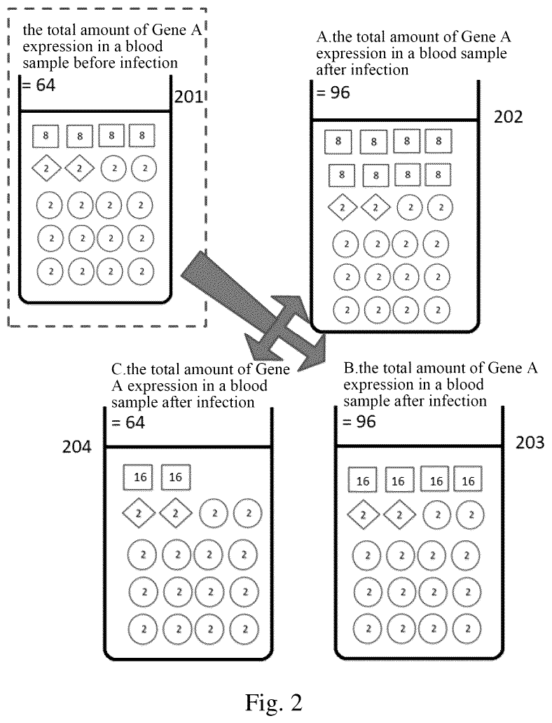

In contrast, in the present application, gene expression of various cell subpopulations in peripheral blood can be directly determined, and used as a biomarker to differentiate various disease groups. The biomarker can indicate which cell subpopulation causes the difference in gene expression. This new biomarker results from changes in the gene expression of one single cell subpopulation, and therefore, is not affected by changes in the proportional counts of cell subpopulations (FIG. 2).

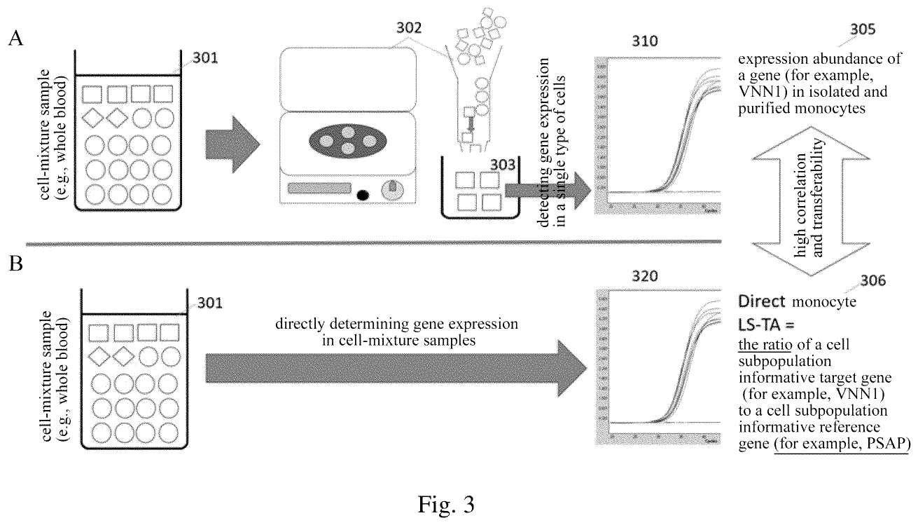

The present application discloses that PSAP or CTSS can be used as the informative reference gene for the Direct Monocyte LS-TA assay of leukocyte subpopulations, and therefore, the expression of target genes specific for one leukocyte subpopulation (for example, monocytes) can be obtained directly from WB or PBMC samples. The cumbersome cell isolation steps are omitted from the detection process, so that this technology can be widely applied in clinical tests (FIG. 3).

Most of the previous biomarkers for differentiation are obtained by comparison of two groups, such as control group and disease group, bacterial infection group and viral infection group, latent tuberculosis disease and active pulmonary tuberculosis disease. In contrast, in the present application, two or more Direct Monocyte LS-TA biomarkers can be used to differentiate multiple groups of diseases (i.e. simultaneously differentiating bacterial infections, viral infections, active pulmonary tuberculosis, and autoimmune diseases) (see Example 7).

In addition, MoM is used in the present application as a region for group classification and differentiation by biomarkers. In general, a normal reference region of the control group is used by the biomarker. If a sample is outside the normal reference region, it is defined as a disease or abnormality. In the present application, the median expression value (median of control group) of each Direct Monocyte LS-TA in the normal group is first defined, and then the value of a test sample is expressed as a multiple (folds) of the median of the normal group (multiple of median of control group, MoM). MoM is used to delineate differentiation boundaries and differentiating regions and intervals for different groups. The MoM used in the present application solves the problem that values generated by different assay platforms (such as various microarrays or RNA-seq) cannot be converted to each other in the past. The use of MoM effectively solves the problem of exchange of results between assay platforms. The MoM grouping differentiation regions and intervals in the present application can be implemented across different assay platforms (e.g., microarray and RNA-seq).

### Terms and Definitions

The term “direct measurement” as used herein refers to a measurement without isolating the specific cells (e.g., monocytes). That is, the direct measurement is performed on a blood sample without isolating the specific cells to be determined (e.g., monocytes) therefrom.

The term “cell-mixture sample” as used herein is a mixture of cells obtained from an individual (such as a human). Typically, the cell-mixture sample may be obtained from peripheral blood, and may be, e.g., a peripheral blood sample without any prior manipulation.

The term “peripheral blood sample(s)” as used herein generally includes whole blood samples (WB) and peripheral blood mononuclear cell samples (PBMCs), both of which are cell-mixture samples and contain various leukocyte subpopulations. In various types of peripheral blood samples, cell counts and proportional cell counts of various leukocytes may vary.

The term “peripheral blood mononuclear cells (PBMCs)” as used herein is a peripheral blood sample, in which there are various mononuclear leukocyte subpopulations, including lymphocytes and monocytes. The primary method for isolating peripheral blood mononuclear cells is Ficoll-hypaque density gradient centrifugation.

The term “leukocyte subpopulation (LS)” as used herein includes many types of cells, also known as cell subpopulations. Peripheral blood is a typical cell-mixture sample with multiple types of cells, including various leukocyte subpopulations, such as neutrophils, lymphocytes, and monocytes.

The term “monocytes” as used herein (also known as mononuclear leukocytes, mononuclear white cells, mononuclear white blood cells, or mononuclear spheres in Chinese Translation) is a cell subpopulation of leukocytes. Monocytes can be found in two common peripheral blood samples, i.e., whole blood samples (WB) and peripheral blood mononuclear cell samples (PBMCs). Monocytes are the largest blood cells in the blood and also the largest leukocytes in volume, and are an important part of the body's defence system. Monocytes are derived from hematopoietic stem cells in bone marrow and developed in bone marrow. Monocytes are still immature cells when they enter the bloodstream from the bone marrow. At present, it is believed that monocytes are the predecessors of macrophages and dendritic cells, have an apparent amoeboid movement and is capable of phagocytosis and removing damaged and aging cells and their debris. Monocytes also participate in immune responses, and after phagocytosis of antigens, they present antigenic determinants to lymphocytes to induce specific immune responses of the lymphocytes. Monocytes are also in the main cellular defence system against intracellular pathogenic bacteria and parasites, and also have the ability to recognize and kill tumour cells. Compared with other blood cells, monocytes contain more non-specific lipases and have stronger phagocytosis. When inflammation or other diseases occur in the body, the percentage of the total number of monocytes will change, and therefore, the examination of monocyte counts becomes a method of auxiliary diagnosis. On the other hand, the expression level of target genes in monocytes can be directly measured in the present application.

The term “biological indicator” and “biomarker” as used herein, also known as an “indicator”, can reflect the amount of messenger ribonucleic acid expressed by or expression level of genes in specific cells (such as monocytes), including a relative amount and an absolute amount which can be used to indicate the status of the biomarker. The biomarker herein refers to the Direct Monocyte LS-TA, and the “biomarker” and “biological indicator” can be used interchangeably. Its values are called biomarker parameters or parameters for short.

The term “transcript abundance (TA)” as used herein refers to the gene expression level obtained by detecting a sample. The term “transcript” as used herein refers to a product from gene transcription, typically an RNA. For example, a protein-coding gene will produce messenger RNA (mRNA). By determining the mRNA amount of this gene, the expression level of the gene (which can also be called “transcript abundance”) can be obtained.

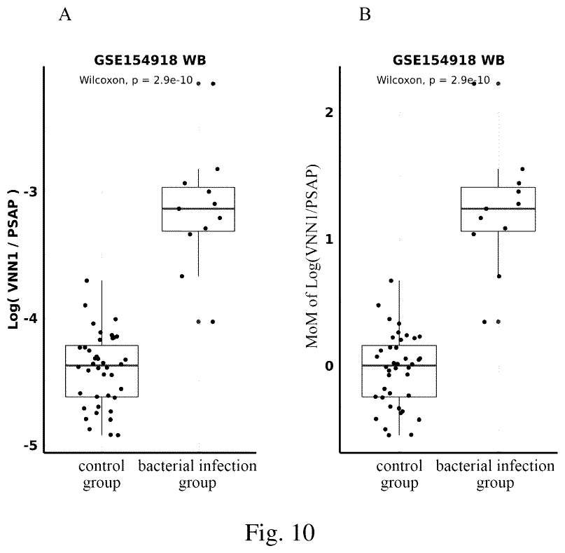

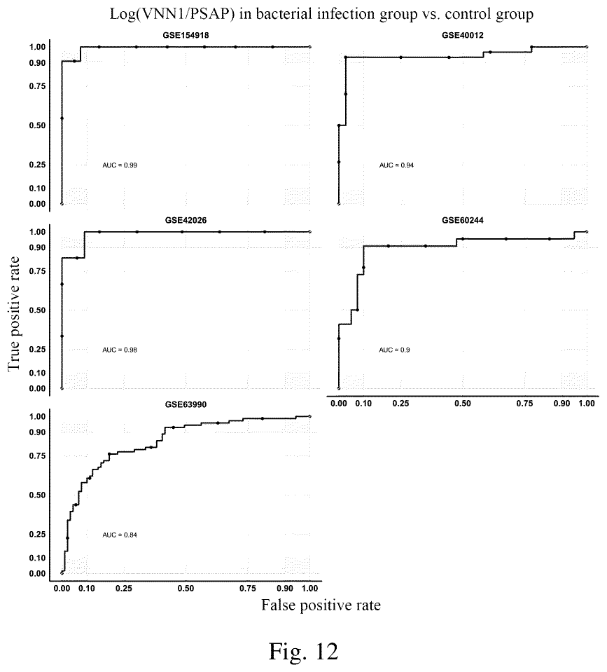

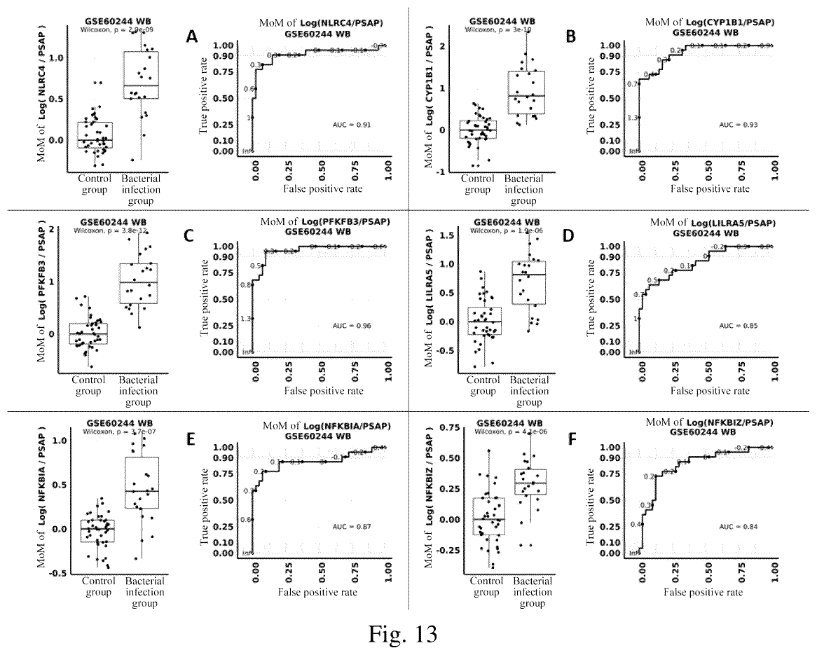

The term “cutoff value” can be defined in several ways. (1) The cutoff value can be defined as a value beyond the reference region of the control group. The reference region of the control group usually takes the distribution of the middle 95% of the control group. A value beyond this range can be used as a cutoff value to define lower outlier or high outlier results. (2) The cutoff value can also be defined from the ROC chart, as shown in FIG. 12. The value marked at each point in the ROC curve represents a potential cutoff value, and the Y-axis and X-axis show the relative sensitivity and specificity using this cut-off value, respectively (FIG. 12). Therefore, when-4 is used as a cutoff value for the direct LS-TA values of the log(VNN1/PSAP) ratio, the sensitivity and specificity for determining bacterial infections in the GSE154918 dataset (upper left panel in FIG. 12, and FIG. 10) are both about 0.9 (upper left panel in FIG. 12). (3) If no control group is available, the percentile value of distribution of the direct LS-TA data in the patient group can also be used as a cutoff value.

The term “Direct Monocyte Subpopulation Transcript Abundance Assay (abbreviated as “Direct Monocyte LS-TA” assay)” as used herein is a novel scheme for biomarker parameter assays, particularly for cell-mixture samples with multiple types of cells, in which the average gene expression of the subpopulation monocytes can be directly assessed from the cell-mixture samples without the need to isolate and purify the subpopulation monocytes therein. The calculation of direct monocyte LS-TA requires the use of cell subpopulation (monocytes) informative target genes and cell subpopulation (monocytes) informative reference genes.

In some embodiments, the Direct Monocyte LS-TA value can be calculated by using the ratio of the cell subpopulation informative target genes to the cell subpopulation informative reference genes. For example, “Direct Monocyte LS-TA” of a certain target gene=(this target gene in PBMCs)/(corresponding reference gene in PBMCs). In other embodiments, log(ratio) is used to calculate the Direct Monocyte LS-TA value. For example, log(“Direct Monocyte LS-TA” of VNN1)=log(VNN1 in PBMCs)−log(PSAP in PBMCs).

The majority (≥50%) of transcripts of the term “leukocyte subpopulation informative genes” (abbreviated as “cell subpopulation informative genes” or “informative genes”) as used herein in a cell-mixture sample with multiple cells (such as WB and PMBC) is from a specified target cell subpopulation, such as monocytes. The cell subpopulation informative genes include target genes and reference genes. In the present application, “characteristic genes” and “informative genes” have the same meaning and can be used interchangeably.

The majority (more than half, >50%) of transcripts of the term “leukocyte subpopulation informative reference genes” (abbreviated as “reference gene”) as used herein in a cell-mixture sample with multiple cells (such as WB and PMBC) is from a specified target cell subpopulation, and meanwhile, expression of the genes in target cells is also relatively stable with low between-individual variances, and the genes are different from the commonly used housekeeping genes. Examples of monocyte subpopulation informative reference genes in the present application include PSAP and CTSS.

The term “subpopulation informative target genes” (also called “subpopulation target genes”, or “target genes”) as used herein are selected from the informative genes of a specified cell subpopulation. These genes may be involved in some target pathways, may be differentially expressed between healthy subjects and patients, or may be co-expressed with other target genes.

The term “bacterial infection” as used herein refers to acute bacterial infection, rather than sepsis which also known as pyemia or septicaemia. Generally, febrile patients encountered in the emergency room or outpatient clinics are in the stage of acute bacterial infection. If there is no appropriate treatment, they will further develop into systemic inflammatory responses, resulting in pyemia and acute organ dysfunction (Singer et al. 2016; Cecconi et al. 2018; Gunsolus et al. 2019). Since pyemia only occurs in some patients with bacterial infection and with strong inflammatory responses, and has its own special gene expression (Miller et al. 2018), it is not the condition of interest in the present application. Therefore, if relevant data are provided in the database, pyemia samples will be filtered out before the calculation is performed.

### List of Datasets Used in the Present Application

### Gene Expression Datasets of Peripheral Blood and Specific Single Cell-Types

To identify monocyte informative genes that are suitable for the direct leukocyte subpopulation transcript abundance assay, various gene expression datasets obtained from peripheral blood samples were used.

These datasets were available from the Gene Expression Omnibus (GEO), maintained by the US National Institutes of Health. Details were available under their accession numbers. The types of peripheral blood samples obtained included whole blood (WB) and peripheral blood mononuclear cells (PBMCs). Specific cell types that has been further isolated and purified, such as isolated and purified monocytes, were also included in some datasets. A list of some exemplary datasets is set forth in Table 1 below.

TABLE 1
An exemplary list of datasets

| Dataset | | Type of peripheral | |
|---|---|---|---|
| accession | | blood sample | |
| number | Usage | (WB or PBMC) | References |
| GSE138746 | 1. To calculate the 90^{th} | PBMC and isolated | (Tao et al. |
| | percentile value of the | and purified | 2021) |
| | fold of expression in | monocytes | |
| | monocytes vs. PBMCs, | | |
| | and find out genes that | | |
| | meet X50; and | | |
| | 2. To examine and | | |
| | determine the | | |
| | correlation between the | | |
| | biomarker of gene | | |
| | expression obtained by | | |
| | using Direct monocyte | | |
| | LS-TA and the gene | | |
| | expression of isolated | | |
| | and purified monocytes | | |
| | (the gold standard) | | |
| GSE114407 | Ditto | PBMC and isolated | (Kuan et al. |
| | | and purified | 2019) |
| | | monocytes | |
| GSE60424 | Ditto | Whole blood and | (Linsley et |
| | | isolated and | al. 2014) |
| | | purified monocytes | |
| GSE107011 | To compare the gene | PBMC and isolated | (Monaco et |
| | expression of | and purified | al. 2019) |
| | granulocytes | monocytes and | |
| | and monocytes | granulocytes | |

In the present specifications and claims, the words “including”, “comprising” and “containing” mean “including but not limited to”, and are not intended to exclude other parts, additives, components, or steps.

It should be understood that features, characteristics, components or steps described in a particular aspect, embodiment or example of the present application can be applied to any other aspect, embodiment or example described herein unless contradictory therewith.

In the following, the technical solutions in the examples of the present application will be clearly and completely described in conjunction with the drawings in the examples of the present application. It is apparent that the described examples are only parts of the examples of the present application, not all of them. The following examples are illustrative only and are not intended to limit the scope of the embodiments in the present application or the scope of the appended claims. All other examples obtained by those of ordinary skill in the art without any creative efforts based on the examples in the present application fall within the protection scope of the present application.

### EXAMPLES

### Example 1: Determination of Monocyte Informative Genes

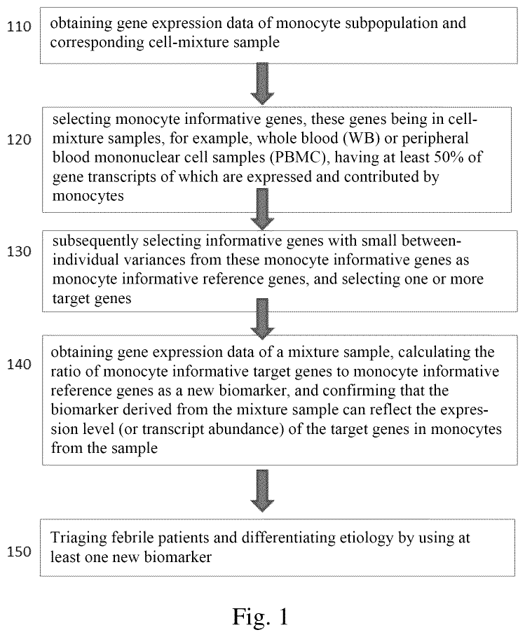

The target blood cell subpopulation of the present application is monocytes. First, we needed to identify monocyte informative genes. Most (≥50%) of gene transcripts of these informative genes in a cell-mixture sample (for example, PBMCs) were produced by single cells (i.e., monocytes). In this example, expression data from the isolated monocyte sample and cell-mixture sample (PBMCs) were used to determine which genes were monocyte informative genes (110 and 120 in FIG. 1).

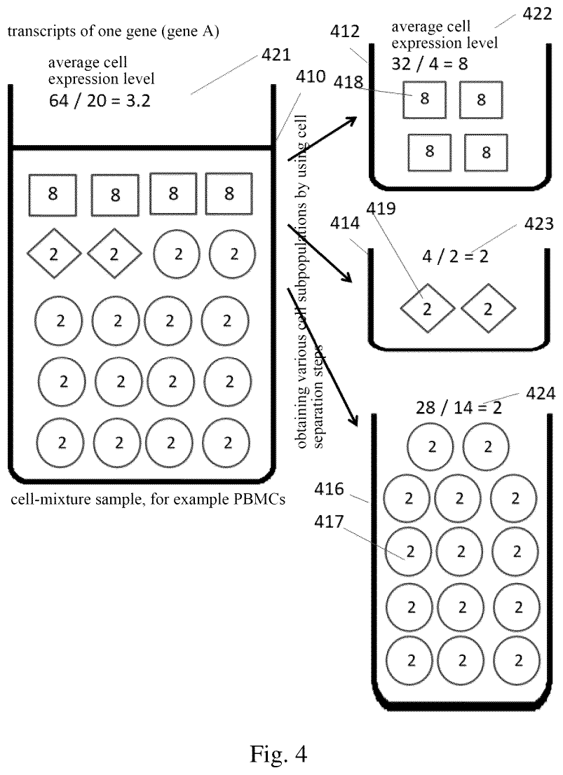

Typically, the cell count percentage of monocytes in PBMCs was 10%-30%. In this example, the cell count percentage of monocytes in PBMCs was set to 20%. As shown in FIG. 4, when the proportional cell count was 20%, the expression of an informative gene in the isolated single cell sample needed to be 2.5 times higher than that in the cell-mixture sample. The fold of this expression difference was designated as X50. Where the target cells were monocytes, X50 was 2.5 times. The monocyte informative genes in the cell-mixture blood sample may be identified by using these conditions.

GSE138746 and other datasets were used in Table 2 below (Tao et al. 2021), where GSE138746 contained 80 paired samples of PBMCs and isolated and purified monocytes from different individuals, 75 of which passed the data quality assessment. The expression data of these two types of samples were used to calculate the fold values of the expression of each gene in monocytes relative to the expression of that in PBMCs, and 90 percentile values were obtained from the range of fold values of 75 individuals. As the X50 required for the monocyte informative genes was ≥2.5x, that is, at least 50% of the gene transcripts in the cell-mixture sample were produced by monocytes, when the 90^{th} percentile value was higher than 2.5, the gene was the monocyte informative gene. Since granulocytes (such as neutrophils) account for the majority of peripheral blood cells, the monocyte informative genes in Table 2 also excluded genes whose expression in granulocytes were higher than that in monocytes. The GSE107011 dataset (Monaco et al. 2019) in Table 1 was used to compare the gene expression in both granulocytes and monocytes.

TABLE 2
List of Monocyte Informative Reference Genes

| | The 90^{th} | | | |
|---|---|---|---|---|
| | percentile | | | |
| | value of the | | | |
| | fold of | | | |
| | expression in | | | |
| | monocytes vs. | | | |
| | peripheral | | | |
| | blood samples | Co- | | |
| | (the fold | efficient | | |
| | required for | of | | |
| Reference | X50 was 2.5 | variation | | |
| genes | or higher) | (CV %) | Database source | |
| CTSS | 2.8 | 9% | GSE138746 | |
| PSAP | 3.2 | 11% | GSE138746 | |
| | | | Correlation between | |
| | | | the Direct Monocyte | |
| | | | LS-TA biomarker | |
| | The 90^{th} | | obtained directly | |
| | percentile | | from peripheral | |
| | value | | blood samples and | |
| | of the fold of | | the gold standard | |
| | expression in | | (expression level | |
| | monocytes vs. | Co- | of target genes in | |
| | peripheral | efficient | isolated and | |
| | blood or | of | purified monocytes) | |
| | PBMCs | variation | (coefficient of | Database |
| Target gene | (X50) | (CV %) | determination, r²) | source |
| ASGR2 | 3.6 | 41% | 0.79 | GSE138746 |
| ATF3 | 3.9 | 164% | 0.64 | GSE138746 |
| CALHM6 | 2.7 | 78% | 0.78 | GSE138746 |
| CD163 | 3.5 | 65% | 0.85 | GSE138746 |
| CD36 | 3.5 | 22% | 0.64 | GSE138746 |
| CDKN1A | 2.8 | 101% | 0.81 | GSE138746 |
| CES1 | 3.7 | 51% | 0.85 | GSE138746 |
| CLEC12A | 3.1 | 33% | 0.73 | GSE138746 |
| CRISPLD2 | 3.8 | 31% | 0.72 | GSE138746 |
| CXCL10 | 3.6 | 212% | 0.84 | GSE60424 |
| CYP1B1 | 3.9 | 45% | 0.80 | GSE138746 |
| CYP27A1 | 4.1 | 30% | 0.65 | GSE138746 |
| EREG | 6.6 | 172% | 0.66 | GSE138746 |
| FOS | 4.3 | 83% | 0.82 | GSE138746 |
| GADD45B | 2.6 | 69% | 0.70 | GSE138746 |
| IER2 | 3.0 | 80% | 0.74 | GSE138746 |
| IFI30 | 3.3 | 20% | 0.60 | GSE60424 |
| IFI44L | 3.2 | 141% | 0.95 | GSE60424 |
| IFITM3 | 2.7 | 100% | 0.87 | GSE138746 |
| IL1B | 3.8 | 183% | 0.80 | GSE138746 |
| KLF10 | 2.6 | 52% | 0.83 | GSE138746 |
| LILRA5 | 3.4 | 23% | 0.55 | GSE114407 |
| LYZ | 3.4 | 23% | 0.69 | GSE138746 |
| MAFB | 4.1 | 65% | 0.73 | GSE138746 |
| MARCO | 3.5 | 38% | 0.75 | GSE138746 |
| MERTK | 3.4 | 45% | 0.60 | GSE138746 |
| MYOF | 2.8 | 50% | 0.68 | GSE138746 |
| NAIP | 3.3 | 45% | 0.66 | GSE138746 |
| NFKBIA | 3.8 | 37% | 0.55 | GSE114407 |
| NFKBIZ | 2.7 | 33% | 0.60 | GSE114407 |
| NLRC4 | 3.0 | 31% | 0.74 | GSE138746 |
| NR4A1 | 2.5 | 149% | 0.82 | GSE138746 |
| NRG1 | 4.4 | 55% | 0.85 | GSE138746 |
| PFKFB3 | 3.2 | 40% | 0.68 | GSE114407 |
| RHOB | 3.8 | 66% | 0.72 | GSE138746 |
| RNF144B | 2.8 | 48% | 0.75 | GSE138746 |
| RPH3A | 4.0 | 66% | 0.73 | GSE138746 |
| SCO2 | 2.9 | 48% | 0.66 | GSE138746 |
| SGK1 | 3.4 | 71% | 0.82 | GSE138746 |
| SHTN1 | 3.6 | 32% | 0.65 | GSE138746 |
| SIGLEC1 | 3.8 | 171% | 0.94 | GSE138746 |
| SULT1A1 | 2.5 | 38% | 0.80 | GSE138746 |
| TCN2 | 4.0 | 51% | 0.65 | GSE138746 |
| TLR7 | 2.6 | 46% | 0.66 | GSE138746 |
| TMEM176A | 3.9 | 73% | 0.92 | GSE138746 |
| TMEM176B | 3.9 | 72% | 0.97 | GSE138746 |
| VNN1 | 4.2 | 63% | 0.80 | GSE138746 |
| WARS1 | 2.5 | 52% | 0.72 | GSE138746 |

### Example 2: Determination of Monocyte Informative Reference Genes

Based on the monocyte informative genes in Table 2, their between-individual variances were calculated and expressed by coefficient of variation (CV %). Genes with low between-individual variances may be used as the monocyte informative reference genes. From the above Table 2, the coefficient of variation (CV %) of CTSS gene and PSAP gene was the smallest, and their CV % are 9% and 11%, respectively. Therefore, the two genes were selected as the monocyte informative reference genes.

In general, CD14 was a known cell membrane protein specific for monocytes and was used to isolate monocytes. Those skilled in the art would attempt to use the CD14 gene as a reference gene, so as to obtain monocyte-specific gene expression indicators in cell-mixture samples (e.g., PBMCs or WB). However, the results showed that the between-individual variance of CD14 was high (CV %=21%), which was more than two times higher than that of the two monocyte informative reference genes (PSAP and CTSS) selected herein.

Here, the performance of using CD14 gene as the reference gene was compared with the performances of PSAP gene and CTSS gene. This comparison requires the use of databases containing gene expression in isolated monocytes and gene expression in cell-mixture samples, using the gene expression level in the isolated monocytes as the gold standard. Then, using different reference genes, the expression parameters of target genes were calculated for the cell-mixture samples, and the parameters calculated for the cell-mixture samples were subsequently compared with the gold standard (correlation or other similar statistical methods may be used for the comparison) to identify which genes were effective monocyte informative reference genes.

As shown in FIG. 6A, when GSE60424 database was used (Linsley et al. 2014), LYZ was selected as the target gene, for its known high expression in monocytes, and CD14 was used as the reference gene in the cell-mixture sample. However, the performance was not satisfactory, with a coefficient of determination (also known as determination coefficient, or determining coefficient, abbreviated as r² (equal to the square of the correlation coefficient)) of only 0.074. That is, using CD14 as the reference gene, only expression variance of 7% of the target gene LYZ of monocytes may be inferred. In contrast, as shown in FIG. 6B, when GSE60424 database was used, and CTSS identified in the present application was used as the reference gene, the new biomarker parameter (LYZ/CTSS) calculated was able to reflect the expression variance of 70% of the target gene LYZ of monocytes (r²=0.7). The above results showed that the performance was improved by ten times by using the new biomarker derived by the reference genes of the present application.

In FIG. 6, the specified target gene was LYZ, and a conventional housekeeping gene was used for isolated monocyte samples to calibrate the total amount of transcripts used in the experiment. The conventional housekeeping gene was selected from Eisenberg and Levanon 2013, and were also available at https://www.tau.ac.il/˜elieis/HKG/. For this example, the conventional housekeeping gene was only used to normalize gene expression results from isolated and purified monocyte samples. Examples of the conventional housekeeping gene included B2M, ACTB, GAPDH and UBC. In FIG. 6, B2M was used as the conventional housekeeping gene. It should be noted that the conventional housekeeping gene was used only by manufacturers for calibration of the gold standard and for verification purposes (e.g., verification of correlation with the gold standard), not for use in the kit or other embodiments of the present application.

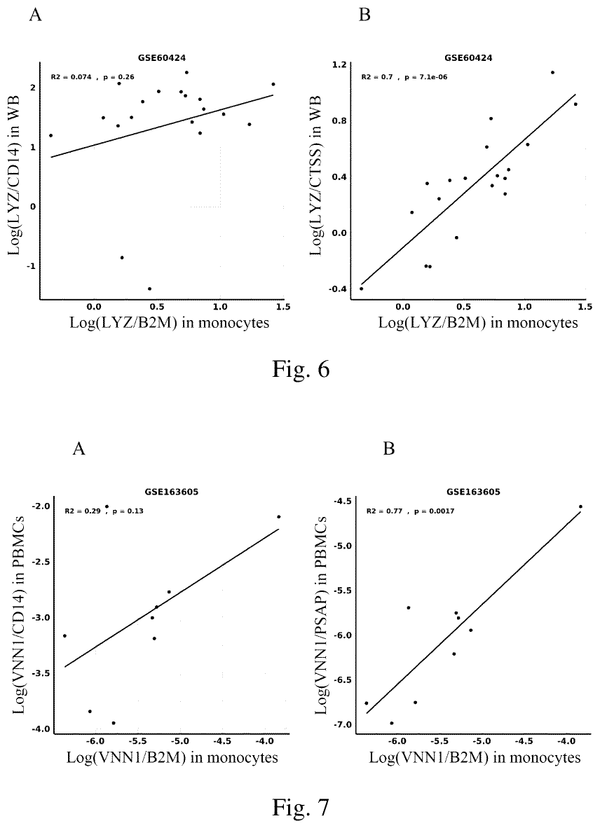

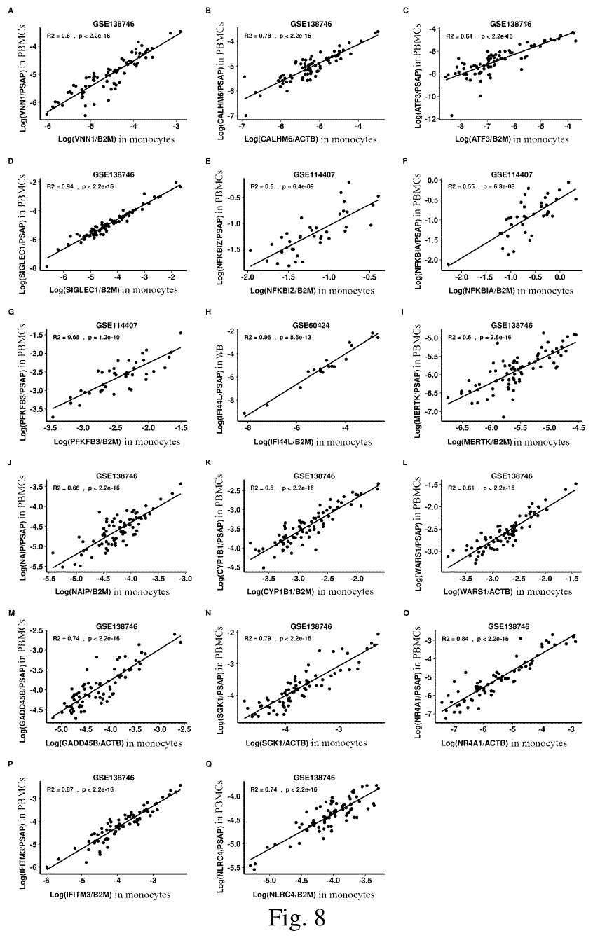

As shown in FIG. 7A, when GSE163605 database was used, VNN1 was used as the target gene, and CD14 was used as the reference gene in the cell-mixture sample, the r² was only 0.29. In contrast, as shown in FIG. 7B, the r² of the direct monocyte LS-TA marker obtained by using the PSAP or CTSS identified in the present application as the monocyte informative reference gene and the gold standard exceeded 0.7.

As shown in FIGS. 6 and 7, the results obtained by using various target genes and databases demonstrated that (1) CD14 was not an ideal monocyte informative reference gene, and (2) PSAP and CTSS were valid monocyte informative reference genes. The direct monocyte LS-TA marker, which was highly consistent with the gene expression of isolated monocytes, may be directly obtained in the mixed sample by the reference genes PSAP and CTSS disclosed in the present application, providing an effective direct monocyte gene expression assay, in which the cumbersome cell isolation step may be omitted.

### Example 3: Biomarker Parameter of “Direct Monocyte Transcript Abundance (Abbreviated as “Direct Monocyte LS-TA”)”

One monocyte subpopulation informative gene (such as VNN1) was selected as a target gene, and the transcript abundance (TA) of the target gene was determined in a cell-mixture sample (such as whole blood). A new biomarker parameter was calculated from the ratio of the transcript abundance (TA) of the target gene to the transcript abundance of another monocyte informative reference gene (such as PSAP or CTSS) to reflect the gene expression level in the isolated and purified monocytes. This new biomarker parameter is called “Direct Monocyte Transcript Abundance” (abbreviated as “Direct Monocyte LS-TA”).

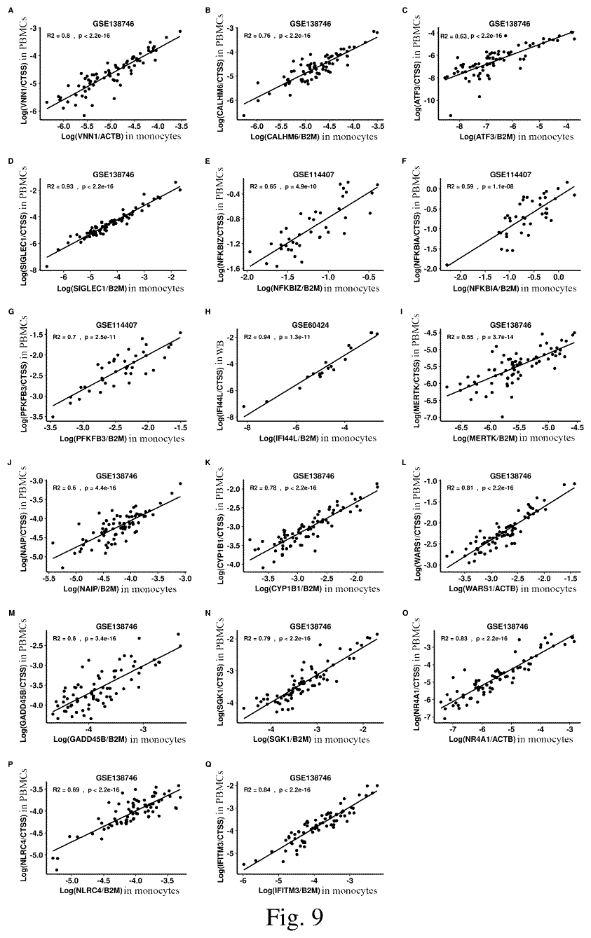

As shown in FIG. 8, in dataset GSE138746, a number of isolated and purified monocyte samples and corresponding PBMC samples were collected. The ratio of two specified monocyte informative genes (i.e., a target gene and a reference gene) in the PBMC samples was called the biomarker parameter of “Direct Monocyte LS-TA”. The correlation between this parameter and the expression of the target gene in the monocyte samples after isolation and purification provided a performance assessment of whether the biomarker in the present application (i.e., Direct Monocyte LS-TA, shown in the Y-axis of FIG. 8) could represent the gene expression of monocytes.

As shown in the Y-axes of FIG. 8, the biomarker parameter of “Direct Monocyte LS-TA” of the present application (for example, the ratio of genes VNN1:PSAP in FIG. 8A) measured in the cell-mixture sample of peripheral blood correlated well with the expression of target genes (for example, target gene VNN1 on the X-axis in FIG. 8A) determined in monocytes after isolation and purification by the traditional method. It was confirmed by the results that the biomarker parameter of “Direct Monocyte LS-TA” determined directly from the peripheral blood samples could be used to assess the expression of target genes (such as the gene VNN1 in FIG. 8A) in purified monocytes, and it was determined that the determination of gene expression in monocytes could be obtained directly from cell-mixture samples (including PBMC or WB) without the need of prior isolation of monocytes. The target genes suitable for “Direct Monocyte LS-TA” included but were not limited to VNN1, CALHM6, ATF3, SIGLEC1, NFKBIZ, NFKBIA, PFKFB3, IFI44L, MERTK, NAIP, CYP1B1, WARS1, GADD45B, SGK1, NR4A1, IFITM3, and NLRC4. The correlation of these target genes with the gold standard were shown in FIG. 8A to FIG. 8Q, respectively.

As shown in FIG. 9, another gene CTSS instead of PSAP was used as the monocyte informative reference gene. The Y-axis of FIG. 9 showed the biomarker parameter of “Direct Monocyte LS-TA” calculated by using the gene CTSS as the denominator (e.g., the ratio of VNN1:CTSS shown in FIG. 9A). The target genes suitable for Direct Monocyte LS-TA included but were not limited to VNN1, CALHM6, ATF3, SIGLEC1, NFKBIZ, NFKBIA, PFKFB3, IFI44L, MERTK, NAIP, CYP1B1, WARS1, GADD45B, SGK1, NR4A1, IFITM3, and NLRC4. The “Direct Monocyte LS-TA” derived by using CTSS as a reference gene also had fairly high correlation with the gold standard, which were shown in FIGS. 9A to 9Q, respectively.

When the reference gene CTSS was applied to different monocyte informative target genes, the performance of the “Direct Monocyte LS-TA” biomarker determined in peripheral blood was almost the same as that when PSAP was used as the reference gene (see FIG. 8 and FIG. 9). Therefore, both PSAP and CTSS genes could be used as valid monocyte informative reference genes.

In the following examples, the “Direct Monocyte LS-TA” marker was used to distinguish several major categories of diseases that lead to fever.

### Example 4: The Use of Multiple of Median (MoM) Enabled the Normalization of the Results of the Biomarker Parameter of “Direct Monocyte LS-TA” Obtained Across Various Databases

In GSE154918 dataset, transcript abundance (TA) of the two specified monocyte informative genes VNN1 and PSAP had been log-transformed (the logarithms used in the present application were natural logarithms). Therefore, log(VNN1) minus log(PSAP) may yield the desired biomarker parameter of the present application (log(VNN1/PSAP) was used in this example). The biomarker parameter represented the expression level of VNN1 gene in monocytes from the cell-mixture samples. As this biomarker parameter may be obtained without the need of prior isolation of monocytes, it was labelled as “Direct Monocyte Transcript Abundance” (abbreviated as “Direct Monocyte LS-TA”, representing Direct Leukocyte Subpopulation Transcript Abundance) in the diagram.

Therefore, the biomarker parameter of the “Direct Monocyte LS-TA” of the monocyte target gene VNN1 may be calculated by using the ratio of the monocyte subpopulation informative target gene and the monocyte subpopulation informative reference gene, and may be expressed as follows:

(“Direct Monocyte LS-TA” of VNN1)=(VNN1 in WB)/(PSAP in WB).

This biomarker parameter may also undergo a logarithmic transformation, and may be expressed as follows:

log(“Direct Monocyte LS-TA” of VNN1)=log(VNN1 in WB)−log(PSAP in WB).

Additionally, since experiments performed with different detection assays would yield results in different units, a method was needed to normalize the results across various datasets obtained from various detection methods. The multiple of median of a normal control group (multiple of median (MoM) of a reference group) was a commonly used normalization method, and the multiple of median of a normal control group may be calculated for the value of each sample. In this example, the direct monocyte transcript abundance determined from the data of the normal control group was used to define the median of control group of the “Direct Monocyte LS-TA”. Results of all individuals (including the diseased group and control group) were then converted to multiples of median of the control group. MOM was often used for detection methods that had not been standardized through large-scale assays, for example prenatal biochemical screening (Driscoll, Gross, and Professional Practice Guidelines Committee 2009), and may be used in cytokine assays for determining the risk of adverse outcomes after SARS-COV infection (Tang et al. 2005). The advantage of using MOM was that it may remove the limitation between datasets due to different detection units among various laboratories, so that comparison may be performed on the results obtained by different detection protocols.

Using the GSE154918 (Herwanto et al. 2021) dataset, the log(“Direct Monocyte LS-TA” of VNN1) (i.e., the log(the ratio of VNN1/PSAP)) was calculated for the samples in the normal control group, the median of which was taken and then subtracted from log(“Direct Monocyte-LS-TA” of VNN1) of all the samples to obtain the MoM of log-transformed “Direct Monocyte-LS-TA” of VNN1 for each sample. This marker reflected the expression abundance of VNN1 gene in monocytes from each whole blood sample.

The sample distribution shown in FIG. 10B had no actual change when compared to the sample distribution in FIG. 10A. The advantage of using the multiple of median (MoM) was that the median of the normal control group was adjusted to zero, which facilitated the comparison of changes in gene expression in disease groups in different databases.

### Example 5: Gene Expression Markers of Monocytes Obtained Directly from Whole Blood Samples Enabled the Differentiation of Bacterial Infections

Table 3 below showed the gene expression datasets used in this example.

TABLE 3
Gene expression datasets used in this example.

| Dataset | | Type of | |
|---|---|---|---|
| accession | Grouping of samples | blood sample | |
| number | and number of samples | (WB or PBMC) | References |
| GSE154918 | Bacterial infection group: 11 | WB | (Herwanto |
| | Control group: 40 | | et al. 2021) |
| | (samples from pyemia | | |
| | patients were not included) | | |
| GSE40012 | Bacterial infection group: | WB | (Parnell |
| | 30 (samples from patients | | et al. 2012) |
| | in bacterial infection | | |
| | group on Day 1 and Day 2) | | |
| | Control group: 36 (samples | | |
| | on Day 1 and Day 5) | | |
| GSE42026 | Bacterial infection group: 18 | WB | (Herberg |
| | Control group: 33 | | et al. 2013) |
| GSE60244 | Bacterial infection group: 22 | WB | (Suarez |
| | Control group: 40 | | et al. 2015) |
| GSE63990 | Bacterial infection group: 71 | WB | (Tsalik |
| | Control group: 89 | | et al. 2016) |

In all datasets, only results from patients with uncomplicated bacterial infection were used, while results from patients with pyemia (if any) were filtered off/removed.

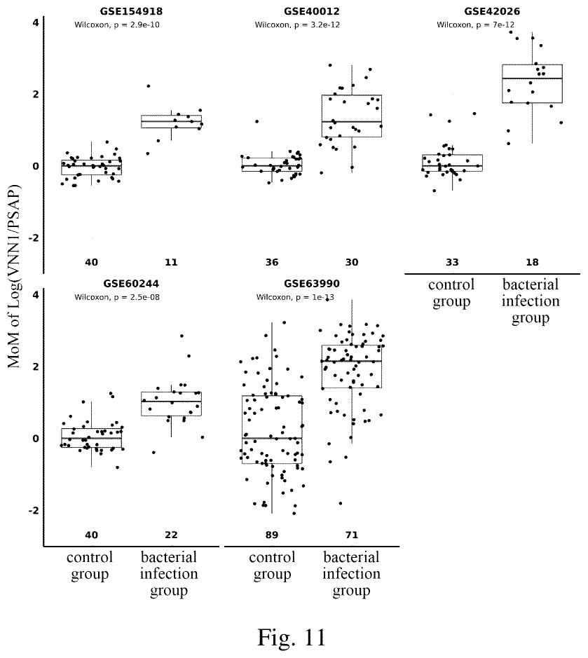

As shown in FIG. 11, in all datasets, there were statistically significant differences in the marker of peripheral blood “Direct Monocyte LS-TA”-target gene VNN1 between the control group and the bacterial infection group (Wilcoxon test, all p values<1e-7). In FIG. 11, it was demonstrated that the “Direct Monocyte LS-TA”-target gene VNN1 was able to differentiate the patients with bacterial infection using various datasets.

Furthermore, receiver operating characteristic curve (ROC) analysis was applied to determine the discrimination ability of “Direct Monocyte LS-TA” of the VNN1 gene for the patients in the bacterial infection group. As shown in FIG. 12, most of the areas under the curve were greater than 0.9, indicating that the “Direct Monocyte LS-TA” of the VNN1 gene had a high discrimination ability for the patients in the bacterial infection group.

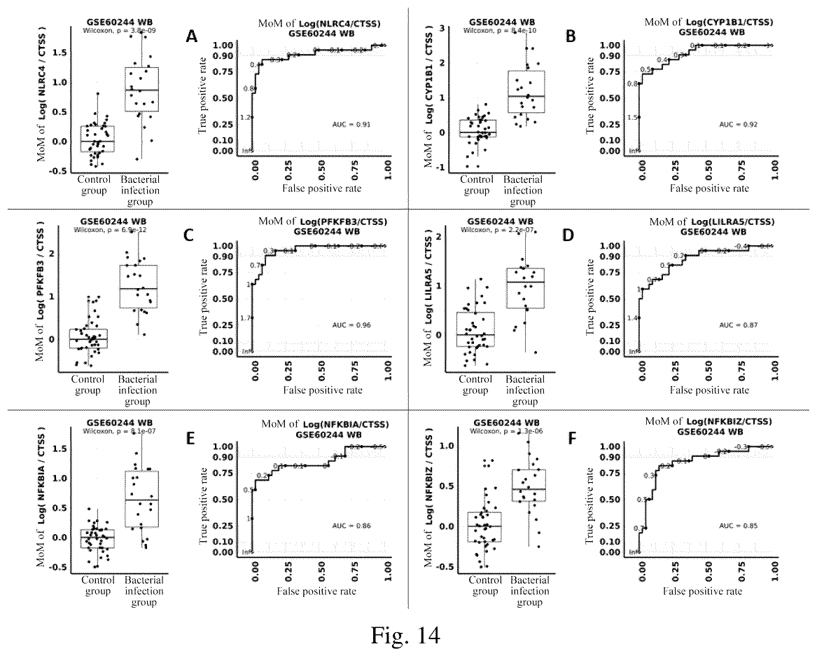

FIGS. 13 and 14 showed additional monocyte informative target genes whose expression was affected by bacterial infections, including NLRC4, CYP1B1, PFKFB3, LILRA5, NFKBIA, and NFKBIZ. These target genes were able to replace or supplement the application of the VNN1 gene in differentiating patients with bacterial infection. There were statistically significant differences in the gene expression of these target genes in peripheral blood mononuclear cells between the control group and the bacterial infection group (Wilcoxon test, all p-values<5e-3). All of the areas under the curve (AUCs) exceeded 0.8. There were no significant differences between the results from the calculation of the Direct Monocyte LS-TA by using PSAP as a reference gene (FIG. 13) and the results from the calculation of the Direct Monocyte LS-TA by using CTSS as a reference gene (FIG. 14), indicating that both PSAP and CTSS may be used as reference genes.

### Example 6: Gene Expression Markers of Monocytes Obtained Directly from Whole Blood Samples (“Direct Monocyte LS-TA”) Enabled the Differentiation of Active Pulmonary Tuberculosis

Table 4 below showed the gene expression datasets used in this example.

TABLE 4
Gene expression datasets used in this example.

| Dataset | | Type of | |
|---|---|---|---|
| accession | | blood sample | |
| | | (WB or | |
| number | Grouping of samples | PBMC) | References |
| GSE107991 | Active pulmonary | WB | (Singhania |
| | tuberculosis group: 21 | | et al. 2018) |
| | Control group: 11 | | |
| GSE107994 | Active pulmonary | WB | (Singhania |
| | tuberculosis group: 50 | | et al. 2018) |
| | Control group: 50 | | |
| GSE114192 | Active pulmonary | WB | (Eckold |
| | tuberculosis group: 149 | | et al. 2021) |
| | Control group: 36 | | |
| GSE42834 | Active pulmonary | WB | (Bloom |
| | tuberculosis group: 40 | | et al. 2013) |
| | Control group: 117 | | |
| GSE83456 | Active pulmonary | WB | (Blankley, Graham, |
| | tuberculosis group: 44 | | Levin, et al. 2016; |
| | Control group: 61 | | Blankley, Graham, |
| | | | Turner, et al. 2016) |

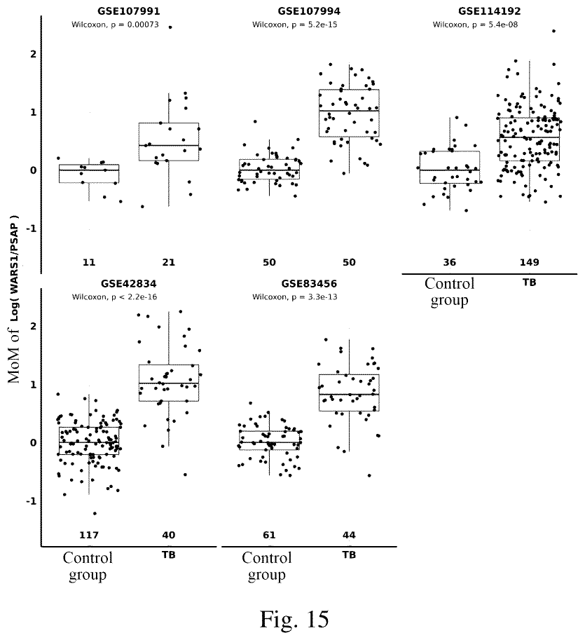

Similar to Example 5, log(“Direct Monocyte LS-TA”-WARS1) and MOM of the log(“Direct Monocyte LS-TA”-WARS1) (FIG. 15, Y axis) of all samples were first calculated for datasets and then the differences between active pulmonary tuberculosis (TB) group and the control group were calculated (Wilcoxon test, p value<0.001) (as shown in FIG. 15). Then, other datasets were used for verification. As shown in FIG. 15, there were statistically significant differences (Wilcoxon test, all p-values<0.001) in the gene expression of peripheral blood mononuclear cells between the control group and the active pulmonary tuberculosis group across all datasets, confirming the direct monocyte LS-TA of the WARS1 gene was significantly increased in patients with active pulmonary tuberculosis.

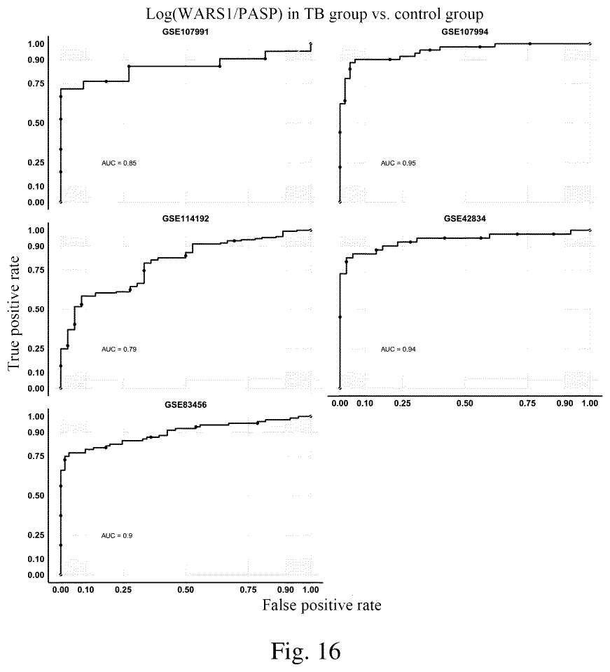

Furthermore, receiver operating characteristic curve (ROC) analysis was applied to determine the discrimination ability of “Direct Monocyte LS-TA” of the WARS1 gene for the patients with active pulmonary tuberculosis. As shown in FIG. 16, most of the areas under the curve were greater than 0.8, indicating that the “Direct Monocyte LS-TA” of the WARS1 gene had a high discrimination ability for the patients with active pulmonary tuberculosis.

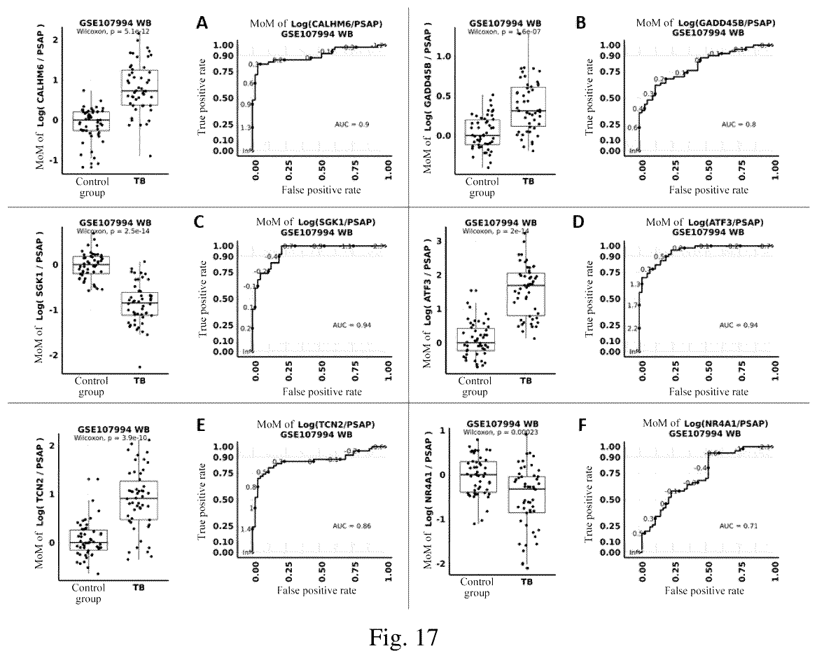

FIG. 17 showed additional monocyte informative target genes whose expression is affected by active pulmonary tuberculosis, including CALHM6, GADD45B, ATF3 and TCN2 with increased gene expression, as well as SGK1 and NR4A1 with decreased gene expression. These target genes were able to replace or supplement the application of the WARS1 gene in differentiating patients with active pulmonary tuberculosis. There were statistically significant differences in the gene expression of these target genes in peripheral blood mononuclear cells between the control group and the active pulmonary tuberculosis group (Wilcoxon test, all p-values<5e-3). Most of the areas under the curve (AUC) exceeded 0.8, indicating that the “Direct Monocyte LS-TA” expressed by the above six genes had a high discrimination ability for the patients with active pulmonary tuberculosis.

### Example 7: Gene Expression Markers of Monocytes Obtained Directly from Whole Blood Samples Enabled the Differentiation of Kawasaki Disease

Kawasaki disease is a common multisystem inflammatory disorder in children. Its clinical symptoms are fever, skin rash, lip erythema, mucosal hyperemia, lesions in lymph nodes, etc. The clinical symptoms of Kawasaki disease highly overlap with those of patients with viral infection, which makes diagnosis difficult. Therefore, a biomarker that can effectively differentiate Kawasaki disease is needed. The inventors found that the gene expression markers (Direct LS-TAs) of monocytes obtained directly from whole blood samples (using one or more of VNN1, CYP1B1, NLRC4, PFKFB3, LILRA5, NFKBIA, NFKBIZ and NAIP genes, and one of two reference genes (PSAP or CTSS)) were able to effectively differentiate Kawasaki disease. The expression of VNN1, CYP1B1, NLRC4, PFKFB3, LILRA5, NFKBIA, NFKBIZ, and NAIP genes was significantly changed in patients with Kawasaki disease. However, changes in the expression of these genes were mild among patients with viral infection. On the contrary, the expression of IFITM3, IFI44L, and IFI30 genes changed significantly. Therefore, the characteristics of these gene expression markers may be used to distinguish Kawasaki disease from viral infections.

It was further demonstrated by using the following data in this example that the gene expression markers of monocytes obtained directly from whole blood enabled efficient differentiation of Kawasaki disease.

Table 5 below showed the gene expression datasets used in this example.

TABLE 5
Gene expression datasets used in this example.

| | | Type of | |
|---|---|---|---|
| | | blood | |
| Dataset | | samples | |
| accession | Grouping of samples | (WB or | |
| number | and number of samples | PBMC) | References |
| GSE73463 | Kawasaki disease group: 141 | WB | (Wright et al. |
| | Control group: 86 | | 2018) |
| GSE73461 | Kawasaki disease group: 75 | WB | (Wright et al. |
| | Control group: 53 | | 2018) |
| GSE68004 | Kawasaki disease group: 87 | WB | (Jaggi et al. |
| | (including classical Kawasaki | | 2018) |
| | disease, complete Kawasaki | | |
| | disease (cKD) and incomplete | | |
| | Kawasaki disease (incomplete | | |
| | presentation, inKD) | | |
| | Control group: 37 | | |

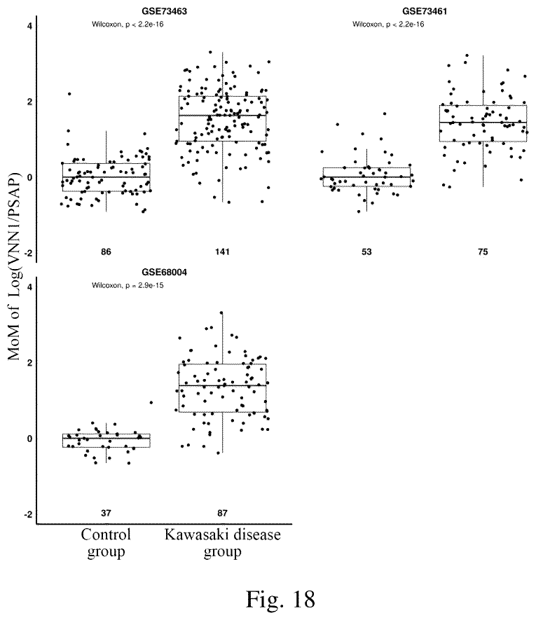

As shown in FIG. 18, across all datasets, there were statistically significant differences in the marker of peripheral blood “Direct Monocyte LS-TA”-target gene VNN1 between the control group and the Kawasaki disease group (Wilcoxon test, all p values<1e-10). In FIG. 18, it was demonstrated that the “Direct Monocyte LS-TA”-target gene VNN1 was able to differentiate patients with Kawasaki disease using various datasets. In contrast, viral infection did not significantly stimulate the marker of peripheral blood “Direct Monocyte LS-TA”-target gene VNN1.

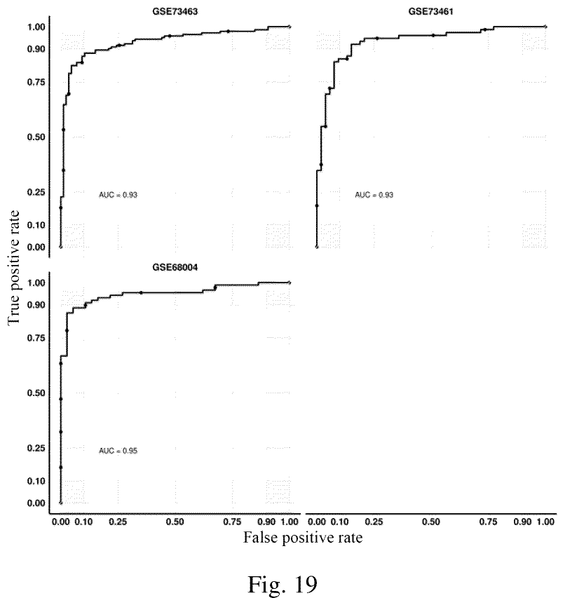

Furthermore, receiver operating characteristic curve (ROC) analysis was applied to determine the discrimination ability of the “Direct Monocyte LS-TA” of the VNN1 gene in patients with Kawasaki disease. As shown in FIG. 19, the areas under the curve for all databases were greater than 0.9, indicating that the “Direct Monocyte LS-TA” of the VNN1 gene had a high discrimination ability in patients with Kawasaki disease.

### Example 8: Two or More Gene Expression Markers of Direct Monocytes Enabled the Differentiation of Febrile-Causing Diseases and Thus May be Used for Patient Triage

Table 6 below showed the gene expression dataset used in this example.

TABLE 6
Gene expression dataset used in this example.

| Dataset | | Type of blood | |
|---|---|---|---|
| accession | | samples (WB | |
| number | Grouping of samples used | or PBMC) | References |
| GSE100150 | Bacterial infection group, | WB | (Altman |
| | Active pulmonary | | et al. 2020) |
| | tuberculosis group, | | |
| | Viral infection group, | | |
| | Systemic lupus | | |
| | erythematosus group | | |
| | (Each disease group | | |
| | had a corresponding | | |
| | control group.) | | |

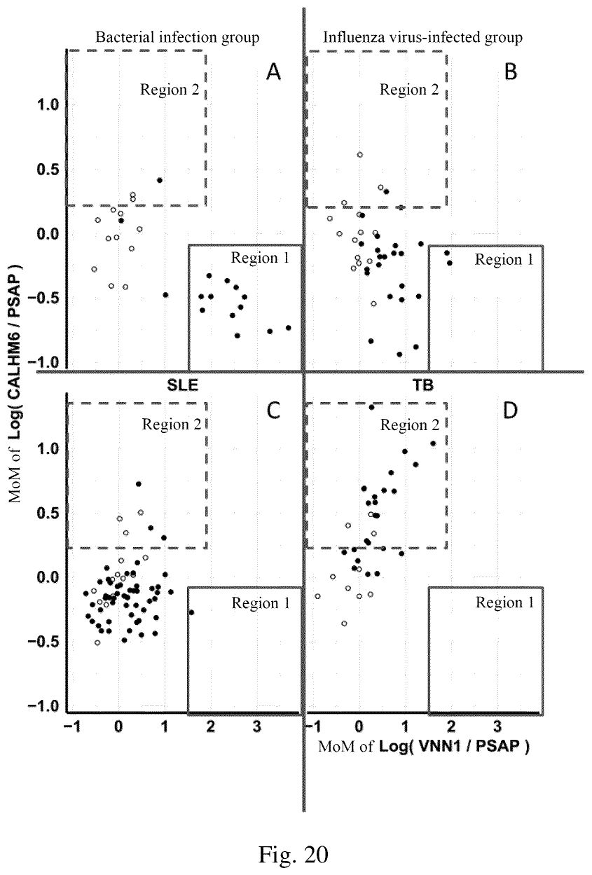

Using database GSE100150 containing multiple types of diseases, the MoMs of two biomarkers, the “Direct Monocyte LS-TA”-VNN1 and “Direct Monocyte LS-TA”-CALHM6, of each sample were obtained by a method similar to that described in Example 6, and were then arranged on a two-dimensional plane to observe the distribution of different types of diseases (FIG. 20). As early antibiotic treatment was needed for bacterial infections, this group was different from the other three disease groups, and here was also included in the sample set of patients in the multiple disease groups to determine whether the above scheme was able to differentiate patients infected with this special group and accuracy therefor.

From the distribution of the MoMs of the “Direct Monocyte LS-TA” of the two genes VNN1 and CALHM6 in patients with bacterial infection, it was apparent that most of the patients with bacterial infection had the characteristics of high Direct Monocyte LS-TA expression of VNN1 (FIG. 20, X-axis) and low Direct Monocyte LS-TA expression of CALHM6 (FIG. 20, Y-axis), and therefore, most of the patients with bacterial infection were distributed in region 1 (see FIG. 20A).

From the distribution of the MoMs of the “Direct Monocyte LS-TA” of these two genes VNN1 and CALHM6 in patients with influenza virus infection (see FIG. 20B) and patients with SLE (see FIG. 20C), it was apparent that only a small number of patients were distributed within these two regions.

From the distribution of the MoMs of the “Direct Monocyte LS-TA” of these two genes VNN1 and CALHM6 in patients with active pulmonary tuberculosis, it was apparent that most of the patients with active pulmonary tuberculosis had the characteristics of low Direct Monocyte LS-TA expression of VNN1 and high Direct Monocyte LS-TA expression of CALHM6, and therefore, most of the patients with active pulmonary tuberculosis were distributed in region 2 (see FIG. 20D).

Based on the results of dataset GSE100150, it may be concluded that there were significant differences in regional distribution between the bacterial infection and active pulmonary tuberculosis groups and the rest of the groups. Different regions may be drawn to differentiate various causes of diseases. As shown in FIG. 20, region 1 may be used to differentiate patients with bacterial infection, region 2 may be used to differentiate patients with active pulmonary tuberculosis, and febrile patients not within these two regions may be infected by viruses or suffer from autoimmune diseases, such as SLE.

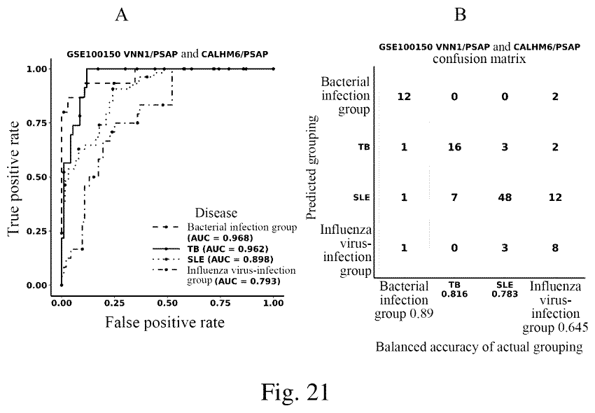

Confusion matrix and ROC analysis were performed by using the MoMs of the “Direct Monocyte LS-TA” of two genes VNN1 and CALHM6 on patients with various causes of fever to determine the discrimination ability of planar distribution of the “Direct Monocyte LS-TA” in the different types of patients (FIG. 21). Firstly, the patients were grouped by a Naïve Bayes Classifier, and then, the grouping results were submitted into a confusion matrix to obtain the balanced accuracy. As the ROC analysis was only suitable for binary classification, here a one-to-many (one vs rest) classification method was used for each type of disease (that is, a certain disease belonged to one category, and other diseases belonged to another category) to obtain the area under the curve (AUC) metric in a ROC analysis corresponding to the diseases. These two metrics may be used to measure the accuracy of differentiation. When the balanced accuracy and the area under the curve were above 0.8, it indicated that the differentiation scheme had a good discrimination ability. As shown in FIG. 21A, the areas under the curve (AUCs) of two patient groups of particular interest (i.e., patients with bacterial infection patients and patients with active pulmonary tuberculosis) were greater than 0.9, and the AUCs of the other two groups of patients, (i.e., patients with influenza virus infection (Flu) and patients with autoimmune disease (SLE)) were also greater than 0.79. As shown in FIG. 21B, the balanced accuracies for patients with bacterial infection, patients with pulmonary tuberculosis, patients with SLE and patients with influenza virus infection were 0.89, 0.816, 0.783, and 0.645, respectively. Therefore, the distribution of the “Direct Monocyte LS-TA” of two genes VNN1 and CALHM6 in two-dimensional space may effectively distinguish the patients with bacterial infection from the patients with pulmonary tuberculosis.

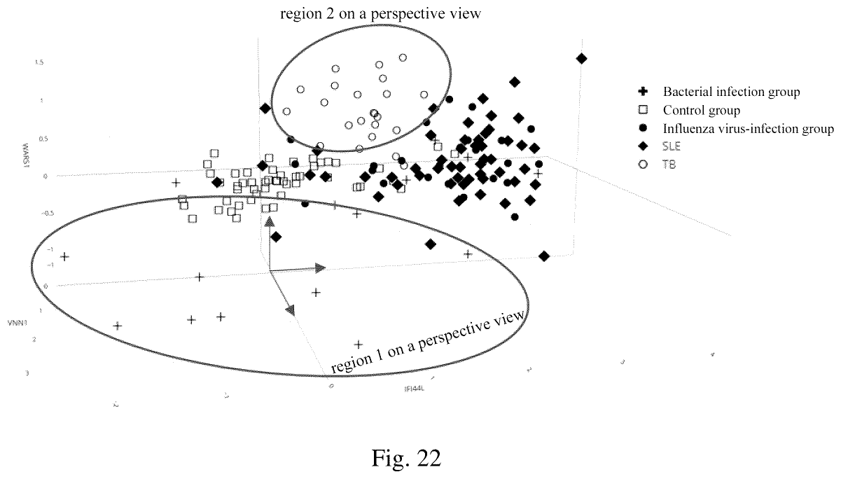

In addition to using the distribution of the MoMs of the “Direct Monocyte LS-TA” of two genes in two-dimensional space, three or more gene expression markers of monocytes (“Direct Monocyte-LS-TA”) may also be projected onto three-dimensional or higher-dimensional space so that patients may be grouped or differentiated. The results showed that in addition to using the distribution of the “Direct Monocyte LS-TA” in two-dimensional space to effectively differentiate different diseases, the discrimination ability of the “Direct Monocyte LS-TA” was also further improved in three-dimensional space (FIG. 22).

FIG. 22 showed the performance of differentiating patients by projecting three gene expression markers of monocytes (MoM of “Direct Monocyte LS-TA”) onto three-dimensional space. The three target genes were VNN1 (FIG. 22, Z axis), WARS1 (FIG. 22, Y axis), and IFI44L (FIG. 22, X axis), and PSAP was used as the direct monocyte informative reference gene. By directly quantification of the ratio of the transcript abundance of the target genes to that of the reference gene (that is, the expression values of a total of four genes) from peripheral blood, the “Direct Monocyte LS-TA” of these three target genes may be obtained, and then compared with the control group to obtain the MoM, so that the three-dimensional spatial distribution plot of FIG. 22 may be plotted. As shown in FIG. 22, when the “Direct Monocyte LS-TAs” of patients with these four diseases were projected onto three-dimensional space, the division between them was more apparent. The patients with bacterial infection (indicated by the symbol “+”) were all distributed in region 1 on the perspective view, the patients with active pulmonary tuberculosis (indicated by the hallow circles) were distributed in region 2 on the perspective view, the patients with influenza virus infection (Flu, indicated by the solid circles) and the patients with autoimmune disease (SLE, indicated by solid diamonds) were mostly outside these two regions, and may also be clearly separated from the normal control group (indicated by hallow squares). The distribution between the groups was more apparent, and was helpful for more accurate differentiation of patients.

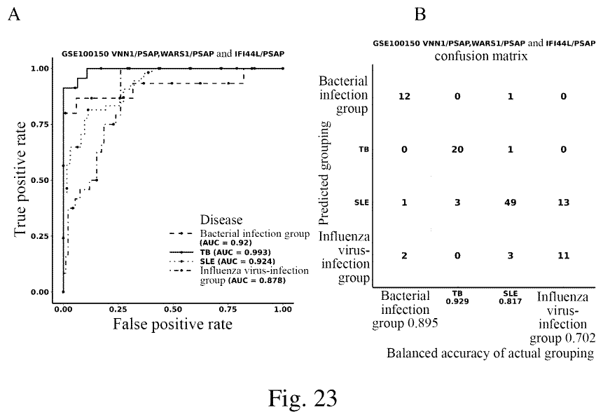

FIG. 23 shows the balanced accuracies obtained by grouping patients using a Naive Bayesian classifier and submitting the grouping results into a confusion matrix. As the ROC analysis was only suitable for binary classification, here a one-to-many (one vs rest) classification method was used for each type of disease (that is, a certain disease belonged to one category, and other diseases belonged to another category) to obtain the area under the curve (AUC) metric in a ROC analysis corresponding to the diseases. These two metrics may be used to measure the accuracy of differentiation. When the balanced accuracy and the area under the curve were above 0.8, it indicated that the differentiation scheme had a good discrimination ability. FIG. 23 showed the results of the confusion matrix and ROC analysis obtained by using the MoM values of the three genes and the Naive Bayes classifier. The areas under the curve (AUCs) of two patient groups of particular interest (i.e., patients with bacterial infection and patients with active pulmonary tuberculosis) were greater than 0.9, and the AUCs of the other two groups of patients, (i.e., patients with influenza virus infection (Flu) and patients with autoimmune disease (SLE)) were 0.924 and 0.878, respectively. As shown in FIG. 23B, the balanced accuracies of the patients with bacterial infection patients, the patients with pulmonary tuberculosis, the patients with SLE patients and the patients with influenza virus infection were 0.895, 0.929, 0.817, and 0.702, respectively. Both had significant improvements over schemes that use two-dimensional spatial distributions.

### Example 9: General Laboratory Procedures for Quantifying Gene Transcript Abundance of Monocytes and Kit Composition

In other embodiments, those skilled in the art will know how to design primers to determine the transcript abundances of these monocyte informative genes.

The present application provides some examples of primers that may be used in quantitative PCR (qPCR) for reference. They may be used in the presence of SYBR Green in qPCR reactions to obtain threshold cycle (CT) data. This data may be used to determine delta-CT, delta-delta CT, or efficiency-corrected delta-CT as biomarker parameters by relative quantification assays. The transcript abundance of RNA in blood samples was quantified (Dorak 2007). Other quantitative methods may also be implemented, including RNA sequencing, DNA microarray (gene chip), branched chain DNA assay (abbreviated as bDNA assay) (see U.S. Pat. No. 8,426,578B2 or 7,927,798B2, which is incorporated herein by reference in its entirety), nanoreporter probe assay (quantification using nanoreporters, see U.S. Pat. No. 8,415,102B2, which is incorporated herein by reference in its entirety), digital PCR (U.S. Pat. No. 10,465,238B2, which is incorporated herein by reference in its entirety) or hybridization.

General laboratory procedures. Firstly, RNA was extracted from various blood samples using Trizol or similar reagents. Commercial kits were also available for column-based RNA extraction. Then, RNA was reversely transcribed into cDNA by reverse transcriptase. Specific genes were quantified by a method of user choice, including qPCR, RNA sequencing, microarray, or hybridization.

The present application teaches that the cDNA sample obtained by this method may be used for the determination of the TAs of monocyte target genes (such as VNN1 or WARS1) and monocyte reference genes (such as PSAP or CTSS), by for example, the primers listed in Table 7. Biomarker parameters would be obtained from delta-CT, delta-delta CT or efficiency-corrected delta-CT of qPCR results for such pair of monocyte informative genes. The biomarker parameters produced by this “Direct Monocyte LS-TA” assay provide an indication of gene expression levels of monocytes in various cell-mixture samples (e.g., PBMC or WB) from blood without the need of prior isolation of monocytes.

TABLE 7
List of examples of primers used for qPCR
assay in the “Direct Monocyte LS-TA” assay.

| Monocyte | | |
|---|---|---|
| informative | Primer 1 | Primer 2 |
| gene | (forward primer) | (reverse primer) |
| VNN1 | 5′-ATTTGGAGGAC | 5′-GGTCTGGCCA |
| | ATCCCAGACC-3′ | AATCTGTTACG-3′ |
| | (SEQ ID NO: 1) | (SEQ ID NO: 2) |
| WARS1 | 5′-CTGTGATGTGG | 5′-CTCCGCTGGT |
| | ACGTGTCTT-3′ | GTAATCCTTC-3′ |
| | (SEQ ID NO: 3) | (SEQ ID NO: 4) |
| PSAP | 5′-ATGGCCGACAT | 5′-GCATGTGCAT |
| | ATGCAAGAA-3′ | CATCATCTGG-3′ |
| | (SEQ ID NO: 5) | (SEQ ID NO: 6) |
| CTSS | 5′-TTCACAACCTG | 5′-TCACTTCTTC |
| | GAGCATTCA-3′ | ACTGGTCATGT-3′ |
| | (SEQ ID NO: 7) | (SEQ ID NO: 8) |

### REFERENCES

[1] CN103764848B Determination of gene expression level of a cell type
[2] U.S. Pat. No. 9,589,099B2 Determination of gene expression levels of a cell type
[3] Altman, Matthew C., Darawan Rinchai, Nicole Baldwin, Mohammed Toufiq, Elizabeth Whalen, Mathieu Garand, Basirudeen Ahamed Kabeer, et al. 2020. “Development and Characterization of a Fixed Repertoire of Blood Transcriptome Modules Based on Co-Expression Patterns Across Immunological States.” https://doi.org/10.1101/525709.
[4] Berry, Matthew P. R., Christine M. Graham, Finlay W. McNab, Zhaohui Xu, Susannah A. A. Bloch, Tolu Oni, Katalin A. Wilkinson, et al. 2010. “An Interferon-Inducible Neutrophil-Driven Blood Transcriptional Signature in Human Tuberculosis.” Nature 466(7309):973-77. https://doi.org/10.1038/nature09247.
[5] Blankley, Simon, Christine M. Graham, Joe Levin, Jacob Turner, Matthew P. R. Berry, Chloe I. Bloom, Zhaohui Xu, et al. 2016. “A 380-Gene Meta-Signature of Active Tuberculosis Compared with Healthy Controls.” The European Respiratory Journal 47(6):1873-76. https://doi.org/10.1183/13993003.02121-2015.
[6] Blankley, Simon, Christine M. Graham, Jacob Turner, Matthew P. R. Berry, Chloe I. Bloom, Zhaohui Xu, Virginia Pascual, et al. 2016. “The Transcriptional Signature of Active Tuberculosis Reflects Symptom Status in Extra-Pulmonary and Pulmonary Tuberculosis.” PloS One 11(10): e0162220. https://doi.org/10.1371/journal.pone.0162220.
[7] Bloom, Chloe I., Christine M. Graham, Matthew P. R. Berry, Fotini Rozakeas, Paul S. Redford, Yuanyuan Wang, Zhaohui Xu, et al. 2013. “Transcriptional Blood Signatures Distinguish Pulmonary Tuberculosis, Pulmonary Sarcoidosis, Pneumonias and Lung Cancers.” PloS One 8(8): e70630. https://doi.org/10.1371/journal.pone.0070630.
[8] Buschmann, Tilo, Sabina CHRIST-BREULMANN, Maik FRIEDRICH, Jens HOHLFELD, Friedemann Horn, Norbert Krug, Kristin Reiche, and Kai Sohn. 2017. Method for the diagnosis of chronic diseases based on monocyte transcriptome analysis. World Intellectual Property Organization WO2017158146A1, filed Mar. 17, 2017, and issued Sep. 21, 2017. https://patents.google.com/patent/WO2017158146A1/en?oq=cst7+biomarker #patentCitations.
[9] Cecconi, Maurizio, Laura Evans, Mitchell Levy, and Andrew Rhodes. 2018. “Sepsis and Septic Shock.” The Lancet 392(10141): 75-87. https://doi.org/10.1016/S0140-6736(18)30696-2.
[10] Chaussabel, Damien. 2015. “Assessment of Immune Status Using Blood Transcriptomics and Potential Implications for Global Health.” Seminars in Immunology 27(1):58-66. https://doi.org/10.1016/j.smim.2015.03.002.
[11] Dorak, M. Tevfik. 2007. Real-Time PCR. Garland Science.
[12] Driscoll, Deborah A., Susan J. Gross, and Professional Practice Guidelines Committee. 2009. “Screening for Fetal Aneuploidy and Neural Tube Defects.” Genetics in Medicine: Official Journal of the American College of Medical Genetics 11(11):818-21. https://doi.org/10.1097/GIM.0b013e3181bb267b.
[13] Eckold, Clare, Vinod Kumar, January Weiner, Bachti Alisjahbana, Anca-Lelia Riza, Katharina Ronacher, Jorge Coronel, et al. 2021. “Impact of Intermediate Hyperglycemia and Diabetes on Immune Dysfunction in Tuberculosis.” Clinical Infectious Diseases: An Official Publication of the Infectious Diseases Society of America 72(1):69-78. https://doi.org/10.1093/cid/ciaa751.
[14] Eisenberg, Eli, and Erez Y. Levanon. 2013. “Human Housekeeping Genes, Revisited.” Trends in Genetics, Human Genetics, 29(10):569-74. https://doi.org/10.1016/j.tig.2013.05.010.
[15] Gliddon, Harriet D., Jethro A. Herberg, Michael Levin, and Myrsini Kaforou. 2018. “Genome-wide Host RNA Signatures of Infectious Diseases: Discovery and Clinical Translation.” Immunology 153(2):171-78. https://doi.org/10.1111/imm.12841.
[16] Gliddon, Harriet D., Myrsini Kaforou, Mary Alikian, Dominic Habgood-Coote, Chenxi Zhou, Tolu Oni, Suzanne T. Anderson, et al. 2021. “Identification of Reduced Host Transcriptomic Signatures for Tuberculosis Disease and Digital PCR-Based Validation and Quantification.” Frontiers in Immunology 12:637164. https://doi.org/10.3389/fimmu.2021.637164.
Gómez-Carballa, Alberto, Ruth Barral-Arca, Miriam Cebey-López, Xabier Bello, Jacobo Pardo-Seco, Federico Martinón-Torres, and Antonio Salas. 2021. “Identification of a Minimal 3-Transcript Signature to Differentiate Viral from Bacterial Infection from Best Genome-Wide Host RNA Biomarkers: A Multi-Cohort Analysis.” International Journal of Molecular Sciences 22(6):3148. https://doi.org/10.3390/ijms22063148.
[18] Gómez-Carballa, Alberto, Miriam Cebey-López, Jacobo Pardo-Seco, Ruth Barral-Arca, Irene Rivero-Calle, Sara Pischedda, María José Currás-Tuala, et al. 2019. “A QPCR Expression Assay of IFI44L Gene Differentiates Viral from Bacterial Infections in Febrile Children.” Scientific Reports 9(1): 11780. https://doi.org/10.1038/s41598-019-48162-9.
[19] Gunsolus, Ian L., Timothy E. Sweeney, Oliver Liesenfeld, and Nathan A. Ledeboer. 2019. “Diagnosing and Managing Sepsis by Probing the Host Response to Infection: Advances, Opportunities, and Challenges.” Journal of Clinical Microbiology 57(7): e00425-19. https://doi.org/10.1128/JCM.00425-19.
[20] Gupta, Rishi K., Carolin T. Turner, Cristina Venturini, Hanif Esmail, Molebogeng X. Rangaka, Andrew Copas, Marc Lipman, Ibrahim Abubakar, and Mahdad Noursadeghi. 2020. “Concise Whole Blood Transcriptional Signatures for Incipient Tuberculosis: A Systematic Review and Patient-Level Pooled Meta-Analysis.” The Lancet. Respiratory Medicine 8(4): 395-406. https://doi.org/10.1016/S2213-2600(19)30282-6.
[21] Herberg, Jethro A., Myrsini Kaforou, Stuart Gormley, Edward R. Sumner, Sanjay Patel, Kelsey D. J. Jones, Stéphane Paulus, et al. 2013. “Transcriptomic Profiling in Childhood H1N1/09 Influenza Reveals Reduced Expression of Protein Synthesis Genes.” The Journal of Infectious Diseases 208(10): 1664-68. https://doi.org/10.1093/infdis/jit348.
[22] Herberg, Jethro A., Myrsini Kaforou, Victoria J. Wright, Hannah Shailes, Hariklia Eleftherohorinou, Clive J. Hoggart, Miriam Cebey-López, et al. 2016. “Diagnostic Test Accuracy of a 2-Transcript Host RNA Signature for Discriminating Bacterial vs Viral Infection in Febrile Children.” JAMA 316(8): 835-45. https://doi.org/10.1001/jama.2016.11236.
[23] Herwanto, Velma, Benjamin Tang, Ya Wang, Maryam Shojaei, Marek Nalos, Amith Shetty, Kevin Lai, Anthony S. McLean, and Klaus Schughart. 2021. “Blood Transcriptome Analysis of Patients with Uncomplicated Bacterial Infection and Sepsis.” BMC Research Notes 14(1): 76. https://doi.org/10.1186/s13104-021-05488-w.
[24] Holcomb, Zachary E., Ephraim L. Tsalik, Christopher W. Woods, and Micah T. McClain. 2017. “Host-Based Peripheral Blood Gene Expression Analysis for Diagnosis of Infectious Diseases.” Journal of Clinical Microbiology 55(2): 360-68. https://doi.org/10.1128/JCM.01057-16.
[25] Kuan, Pei-Fen, Xiaohua Yang, Sean Clouston, Xu Ren, Roman Kotov, Monika Waszczuk, Prashant K. Singh, et al. 2019. “Cell Type-Specific Gene Expression Patterns Associated with Posttraumatic Stress Disorder in World Trade Center Responders.” Translational Psychiatry 9(1): 1. https://doi.org/10.1038/s41398-018-0355-8.
[26] Linsley, Peter S., Cate Speake, Elizabeth Whalen, and Damien Chaussabel. 2014. “Copy Number Loss of the Interferon Gene Cluster in Melanomas Is Linked to Reduced T Cell Infiltrate and Poor Patient Prognosis.” PloS One 9(10): e109760. https://doi.org/10.1371/journal.pone.0109760.
[27] Lydon, Emily C., Ricardo Henao, Thomas W. Burke, Mert Aydin, Bradly P. Nicholson, Seth W. Glickman, Vance G. Fowler, et al. 2019. “Validation of a Host Response Test to Distinguish Bacterial and Viral Respiratory Infection.” EBioMedicine 48(October): 453-61. https://doi.org/10.1016/j.ebiom.2019.09.040.
[28] Mahajan, Prashant, Nathan Kuppermann, Asuncion Mejias, Nicolas Suarez, Damien Chaussabel, T. Charles Casper, Bennett Smith, et al. 2016. “Association of RNA Biosignatures With Bacterial Infections in Febrile Infants Aged 60 Days or Younger.” JAMA 316(8): 846-57. https://doi.org/10.1001/jama.2016.9207.
[29] Mazzone, Massimiliano. 2018. Monocyte biomarkers for cancer detection. U.S. Pat. No. 10,041,126B2, filed Jan. 28, 2013, and issued Aug. 7, 2018. https://patents.google.com/patent/U.S. Pat. No. 10,041,126B2/en?oq=cst7+biomarker.
[30] McClain, Micah T., Florica J. Constantine, Bradly P. Nicholson, Marshall Nichols, Thomas W. Burke, Ricardo Henao, Daphne C. Jones, et al. 2021. “A Blood-Based Host Gene Expression Assay for Early Detection of Respiratory Viral Infection: An Index-Cluster Prospective Cohort Study.” The Lancet. Infectious Diseases 21 (3): 396-404. https://doi.org/10.1016/S1473-3099(20)30486-2.
[31] Mejias, Asuncion, Blerta Dimo, Nicolas M. Suarez, Carla Garcia, M. Carmen Suarez-Arrabal, Tuomas Jartti, Derek Blankenship, et al. 2013. “Whole Blood Gene Expression Profiles to Assess Pathogenesis and Disease Severity in Infants with Respiratory Syncytial Virus Infection.” PLOS Medicine 10(11): e1001549. https://doi.org/10.1371/journal.pmed.1001549.
[32] Miller, Russell R., Bert K. Lopansri, John P. Burke, Mitchell Levy, Steven Opal, Richard E. Rothman, Franco R. D'Alessio, et al. 2018. “Validation of a Host Response Assay, SeptiCyte LAB, for Discriminating Sepsis from Systemic Inflammatory Response Syndrome in the ICU.” American Journal of Respiratory and Critical Care Medicine 198(7): 903-13. https://doi.org/10.1164/rccm.201712-2472OC.
[33] Monaco, Gianni, Bernett Lee, Weili Xu, Seri Mustafah, You Yi Hwang, Christophe Carré, Nicolas Burdin, et al. 2019. “RNA-Seq Signatures Normalized by MRNA Abundance Allow Absolute Deconvolution of Human Immune Cell Types.” Cell Reports 26(6): 1627-1640.e7. https://doi.org/10.1016/j.celrep.2019.01.041.
[34] Nadel, Brian B., Meritxell Oliva, Benjamin L. Shou, Keith Mitchell, Feiyang Ma, Dennis J. Montoya, Alice Mouton, et al. 2021. “Systematic Evaluation of Transcriptomics-Based Deconvolution Methods and References Using Thousands of Clinical Samples.” Briefings in Bioinformatics, August, bbab265. https://doi.org/10.1093/bib/bbab265.
[35] Newman, Aaron M., Chih Long Liu, Michael R. Green, Andrew J. Gentles, Weiguo Feng, Yue Xu, Chuong D. Hoang, Maximilian Diehn, and Ash A. Alizadeh. 2015. “Robust Enumeration of Cell Subsets from Tissue Expression Profiles.” Nature Methods 12(5): 453-57. https://doi.org/10.1038/nmeth.3337.
[36] Parnell, Grant P., Anthony S. McLean, David R. Booth, Nicola J. Armstrong, Marek Nalos, Stephen J. Huang, Jan Manak, et al. 2012. “A Distinct Influenza Infection Signature in the Blood Transcriptome of Patients with Severe Community-Acquired Pneumonia.” Critical Care (London, England) 16(4): R157. https://doi.org/10.1186/cc11477.
[37] Rinchai, Darawan, Jessica Roelands, Mohammed Toufiq, Wouter Hendrickx, Matthew C. Altman, Davide Bedognetti, and Damien Chaussabel. 2021. “BloodGen3Module: Blood Transcriptional Module Repertoire Analysis and Visualization Using R.” Bioinformatics (Oxford, England), February. https://doi.org/10.1093/bioinformatics/btab121.
[38] Sampson, D. L., B. A. Fox, T. D. Yager, S. Bhide, S. Cermelli, L. C. McHugh, T. A. Seldon, et al. 2017. “A Four-Biomarker Blood Signature Discriminates Systemic Inflammation Due to Viral Infection Versus Other Etiologies.” Scientific Reports 7(1): 2914. https://doi.org/10.1038/s41598-017-02325-8.
[39] Shen-Orr, Shai S., Robert Tibshirani, Purvesh Khatri, Dale L. Bodian, Frank Staedtler, Nicholas M. Perry, Trevor Hastie, Minnie M. Sarwal, Mark M. Davis, and Atul J. Butte. 2010. “Cell Type-Specific Gene Expression Differences in Complex Tissues.” Nature Methods 7(4): 287-89. https://doi.org/10.1038/nmeth.1439.
[40] Singer, Mervyn, Clifford S. Deutschman, Christopher Warren Seymour, Manu Shankar-Hari, Djillali Annane, Michael Bauer, Rinaldo Bellomo, et al. 2016. “The Third International Consensus Definitions for Sepsis and Septic Shock (Sepsis-3).” JAMA 315(8): 801-10. https://doi.org/10.1001/jama.2016.0287.
[41] Singhania, Akul, Raman Verma, Christine M. Graham, Jo Lee, Trang Tran, Matthew Richardson, Patrick Lecine, et al. 2018. “A Modular Transcriptional Signature Identifies Phenotypic Heterogeneity of Human Tuberculosis Infection.” Nature Communications 9(1): 2308. https://doi.org/10.1038/s41467-018-04579-w.
[42] Suarez, Nicolas M., Eleonora Bunsow, Ann R. Falsey, Edward E. Walsh, Asuncion Mejias, and Octavio Ramilo. 2015. “Superiority of Transcriptional Profiling over Procalcitonin for Distinguishing Bacterial from Viral Lower Respiratory Tract Infections in Hospitalized Adults.” The Journal of Infectious Diseases 212(2): 213-22. https://doi.org/10.1093/infdis/jiv047.
[43] Sweeney, Timothy E., Hector R. Wong, and Purvesh Khatri. 2016. “Robust Classification of Bacterial and Viral Infections via Integrated Host Gene Expression Diagnostics.” Science Translational Medicine 8(346): 346ra91-346ra91. https://doi.org/10.1126/scitranslmed.aaf7165.
[44] Tang, Nelson Leung-Sang, Paul Kay-Sheung Chan, Chun-Kwok Wong, Ka-Fai To, Alan Ka-Lun Wu, Ying-Man Sung, David Shu-Cheong Hui, Joseph Jao-Yiu Sung, and Christopher Wai-Kei Lam. 2005. “Early Enhanced Expression of Interferon-Inducible Protein-10(CXCL-10) and Other Chemokines Predicts Adverse Outcome in Severe Acute Respiratory Syndrome.” Clinical Chemistry 51(12): 2333-40. https://doi.org/10.1373/clinchem.2005.054460.
[45] Tao, Weiyang, Arno N. Concepcion, Marieke Vianen, Anne C. A. Marijnissen, Floris P. G. J. Lafeber, Timothy R. D. J. Radstake, and Aridaman Pandit. 2021. “Multiomics and Machine Learning Accurately Predict Clinical Response to Adalimumab and Etanercept Therapy in Patients With Rheumatoid Arthritis.” Arthritis & Rheumatology (Hoboken, N.J.) 73(2): 212-22. https://doi.org/10.1002/art.41516.
[46] Tsalik, Ephraim L., Ricardo Henao, Marshall Nichols, Thomas Burke, Emily R. Ko, Micah T. McClain, Lori L. Hudson, et al. 2016. “Host Gene Expression Classifiers Diagnose Acute Respiratory Illness Etiology.” Science Translational Medicine 8(322): 322ra11. https://doi.org/10.1126/scitranslmed.aad6873.
[47] Tsao, Yu-Ting, Yao-Hung Tsai, Wan-Ting Liao, Ching-Ju Shen, Ching-Fen Shen, and Chao-Min Cheng. 2020. “Differential Markers of Bacterial and Viral Infections in Children for Point-of-Care Testing.” Trends in Molecular Medicine 26(12): 1118-32. https://doi.org/10.1016/j.molmed.2020.09.004.
[48] Zaas, Aimee K., Thomas Burke, Minhua Chen, Micah McClain, Bradly Nicholson, Timothy Veldman, Ephraim L. Tsalik, et al. 2013. “A Host-Based RT-PCR Gene Expression Signature to Identify Acute Respiratory Viral Infection.” Science Translational Medicine 5(203): 203ra126. https://doi.org/10.1126/scitranslmed.3006280.
[49] U.S. Pat. No. 8,426,578B2
[50] U.S. Pat. No. 7,927,798B2
[51] U.S. Pat. No. 8,415,102B2
[52] U.S. Pat. No. 10,465,238B2
[53] Jaggi, Preeti, Asuncion Mejias, Zhaohui Xu, Han Yin, Melissa Moore-Clingenpeel, Bennett Smith, Jane C. Burns, et al. 2018. “Whole Blood Transcriptional Profiles as a Prognostic Tool in Complete and Incomplete Kawasaki Disease.” PloS One 13 (5): e0197858. https://doi.org/10.1371/journal.pone.0197858.
[54] Wright, Victoria J., Jethro A. Herberg, Myrsini Kaforou, Chisato Shimizu, Hariklia Eleftherohorinou, Hannah Shailes, Anouk M. Barendregt, et al. 2018. “Diagnosis of Kawasaki Disease Using a Minimal Whole-Blood Gene Expression Signature.” JAMA Pediatrics 172 (10): e182293. https://doi.org/10.1001/jamapediatrics.2018.2293.

1. A method for analyzing a peripheral blood sample, comprising measuring, in the peripheral blood sample, the transcript abundance of a single cell subpopulation target gene and the transcript abundance of a single cell subpopulation reference gene, wherein the single cell subpopulation target gene is selected from at least one of those shown in Table 2-2, and the single cell subpopulation reference gene is selected from PSAP or CTSS, and preferably, the single cell subpopulation is monocytes.

2. Use of a reagent component for measuring the transcript abundance of genes in the preparation of a kit for use in a method for analyzing a peripheral blood sample, wherein the method comprises measuring, in the peripheral blood sample, the transcript abundance of a single cell subpopulation target gene and the transcript abundance of a single cell subpopulation reference gene, and wherein the single cell subpopulation target gene is selected from at least one of those shown in Table 2-2, and the single cell subpopulation reference gene is selected from PSAP or CTSS, and preferably, the single cell subpopulation is monocytes.

3. The method according to claim 1, wherein the single cell subpopulation target gene is selected from one or more of VNN1, CYP1B1, NLRC4, PFKFB3, LILRA5, NFKBIZ, CALHM6, WARS1, ATF3, IFITM3, IFI44L and IFI30.

4. The method according to claim 1, wherein the method comprises the following steps of:
a). obtaining the peripheral blood sample;
b). measuring the transcript abundance of the single cell subpopulation target gene in the peripheral blood sample to obtain a first amount;
c). measuring the transcript abundance of the single cell subpopulation reference gene in the peripheral blood sample to obtain a second amount; and
d). calculating a biomarker parameter, wherein said parameter is a relative value of said first amount to said second amount, and optionally, the method further comprises comparing the relative value to a cutoff value.

5. A kit, comprising a reagent component for quantifying the transcript abundance of genes, wherein the genes are selected from one or more of target genes shown in Table 2-2 and one or more of reference genes shown in Table 2-1.

6. Use of a reagent component for quantifying the transcript abundance of genes as defined in claim 5 in the preparation of a kit or medicament for differentiating and triaging a patient having abnormal body temperature, wherein the genes are selected from one or more of target genes shown in Table 2-2 and one or more of reference genes shown in Table 2-1.

7. The kit according to claim 5, wherein the target genes are a combination of at least one gene selected from group (1) of genes and at least one gene selected from group (2) of genes; a combination of at least one gene selected from group (1) of genes and at least one gene selected from group (3) of genes; a combination of at least one gene selected from group (2) of genes and at least one gene selected from group (3) of genes; or a combination of at least one gene selected from group (1) of genes, at least one gene selected from group (2) of genes and at least one gene selected from group (3) of genes:
(1) VNN1, CYP1B1, NLRC4, PFKFB3, LILRA5, NFKBIA, NFKBIZ and NAIP;
(2) CALHM6, WARS1, GADD45B, NR4A1, SGK1, ATF3 and TCN2; and
(3) IFITM3, IFI44L and IFI30; and
preferably, the target genes are a combination of VNN1 and CALHM6, or the target genes are a combination of VNN1, WARS1 and IFI44L.

8. The use according to claim 6, wherein the patient having abnormal body temperature is a febrile patient, and preferably, the patient having abnormal body temperature is a patient with a bacterial infection, a patient with a viral infection, a patient with a pulmonary tuberculosis or a patient with an autoimmune disease, and more preferably, the patient with a viral infection is a patient with an influenza virus infection, the patient with a pulmonary tuberculosis is a patient with active pulmonary tuberculosis, and the patient with an autoimmune disease is a patient with systemic lupus erythematosus.

9. The use according to claim 7, wherein one or more genes in group (1) of genes are used to differentiate the patient with a bacterial infection, one or more genes in group (2) of genes are used to differentiate the patient with a pulmonary tuberculosis, in particular the patient with active pulmonary tuberculosis, and/or one or more genes in group (3) of genes are used to differentiate the patient with a viral infection or the patient with an autoimmune disease.

10. The use according to claim 7, wherein the patient having abnormal body temperature is a febrile patient, and preferably, the patient having abnormal body temperature is a patient with Kawasaki disease, and wherein one or more genes in group (1) of genes are used to differentiate the patient with Kawasaki disease.

11. The kit according to claim 5, wherein the reagent component comprises primers, and the sequences of the primers are set forth in any one of SEQ ID NOs: 1-8; and preferably, the genes are originated from a peripheral blood sample, more preferably from monocytes in the peripheral blood sample.

12. The use according to claim 2, wherein the single cell subpopulation target gene is selected from one or more of VNN1, CYP1B1, NLRC4, PFKFB3, LILRA5, NFKBIZ, CALHM6, WARS1, ATF3, IFITM3, IFI44L and IFI30.

13. The use according to claim 2, wherein the method comprises the following steps of:
a). obtaining the peripheral blood sample;
b). measuring the transcript abundance of the single cell subpopulation target gene in the peripheral blood sample to obtain a first amount;
c). measuring the transcript abundance of the single cell subpopulation reference gene in the peripheral blood sample to obtain a second amount; and
d). calculating a biomarker parameter, wherein said parameter is a relative value of said first amount to said second amount, and optionally, the method further comprises comparing the relative value to a cutoff value.

14. The use according to claim 6, wherein the target genes are a combination of at least one gene selected from group (1) of genes and at least one gene selected from group (2) of genes; a combination of at least one gene selected from group (1) of genes and at least one gene selected from group (3) of genes; a combination of at least one gene selected from group (2) of genes and at least one gene selected from group (3) of genes; or a combination of at least one gene selected from group (1) of genes, at least one gene selected from group (2) of genes and at least one gene selected from group (3) of genes:
(1) VNN1, CYP1B1, NLRC4, PFKFB3, LILRA5, NFKBIA, NFKBIZ and NAIP;
(2) CALHM6, WARS1, GADD45B, NR4A1, SGK1, ATF3 and TCN2; and
(3) IFITM3, IFI44L and IFI30; and
preferably, the target genes are a combination of VNN1 and CALHM6, or the target genes are a combination of VNN1, WARS1 and IFI44L.

15. The use according to claim 6, wherein the reagent component comprises primers, and the sequences of the primers are set forth in any one of SEQ ID NOs: 1-8; and preferably, the genes are originated from a peripheral blood sample, more preferably from monocytes in the peripheral blood sample.
